(19) United States
(12) Patent Application Publication
(10) Pub. No.: US 2026/0191806 A1
(43) Pub. Date: Jul. 9, 2026

(54) USES OF COMPOSITIONS COMPRISING AN ACID AND AN ALCOHOL

(71) Applicant: Heal Rapidly, LLC, Newport, OR (US)

(72) Inventor: Thomas William Konowalchuk, Newport, OR (US)

(21) Appl. No.: 19/438,483

(22) Filed: Dec. 31, 2025

Related U.S. Application Data
(60) Provisional application No. 63/742,140, filed on Jan. 6, 2025.

(51) Int. Cl. A61K 31/191; A61K 31/045; A61P 17/02; A61P 29/00

(52) U.S. Cl. CPC A61K 31/191; A61K 31/045; A61P 17/02; A61P 29/00

(57) ABSTRACT

The present disclosure provides compositions and use thereof to heal, maintain, or restore the integrity and/or function of the body's surfaces. In particular, the invention provides acidic compositions comprising one or more alcohols and one or more acids. The disclosed compositions and methods are useful in the prevention, treatment, and/or amelioration of a symptom of lesions, inflammation, irritation, infection, and pain of a body surface.

### CROSS-REFERENCE TO RELATED APPLICATION

This application claims the benefit of U.S. Provisional Application No. 63/742,140, filed Jan. 6, 2025, the disclosure of which is incorporated herein by reference.

### FIELD

The invention generally relates to therapeutic compositions and related methods of use to heal, maintain, or restore the integrity and/or function of the internal or external surface structures of plants and animals. In particular, the invention relates to acidic compositions for the prevention, treatment, and/or amelioration of a symptom of lesions, inflammation, irritation, infection, and pain.

### DESCRIPTION OF RELATED ART

The integumentary system is the organ system that protects the body from various kinds of damage, such as loss of water or abrasion from outside. The system comprises the skin and its appendages (including hair, scales, feathers, hooves, and nails). The integumentary system has a variety of functions; it may serve to waterproof, cushion, and protect the deeper tissues, excrete wastes, and regulate temperature, and is the attachment site for sensory receptors to detect pain, sensation, pressure, and temperature.

A mucous membrane is a biological membrane that lines various cavities in the body such as the hollow organs of the respiratory, digestive, urinary, and reproductive tracts and surrounds internal organs. It is of endodermal origin and consists of a layer (or layers) of epithelial cells overlying a layer of loose connective tissue. It is continuous with the skin at various body openings such as the eyes, ears, nose and mouth, the urethral opening and the anus. Some mucous membranes secrete mucus, a thick protective fluid. The function of the membrane is to stop pathogens and dirt from entering the body and to prevent bodily tissues from becoming dehydrated.

Lesions in the human integumentary system and mucous membranes exhibit a wide range of sizes, shapes, colors, penetration depths as well as a wide variety of causes. In the course of healing, adverse effects such as inflammation, irritation, infection, and pain at the site of the lesion or the area proximal to the lesion may occur.

Many pharmaceutical compositions have been used to treat the adverse effects of lesions of the human integumentary system and mucous membranes and to try to speed the healing process with various degrees of efficacy. Frequently, different compositions are used to treat the same type of lesion without scientific merit to support their use. Lacking is an over-the-counter composition that has broad efficacy and low side effects.

### SUMMARY

In various embodiments, a method of decreasing the adverse effects of a lesion or the healing of a lesion, or speeding the reduction of a lesion or decreasing a lesion where the lesion occurs on a surface of a subject or on the surface lining of an internal organ of a subject is provided comprising contacting the lesion and/or an area of the surface adjacent to the lesion with an effective amount of an acidic composition comprising a C1-C10 alcohol, wherein the lesion is not caused by a virus.

In one embodiment, the acidic composition comprises an amount of an acid sufficient to provide a pH of less than 7, such as less than 6.5, less than 6 or less than 5.5. In one embodiment, the acidic composition comprises an amount of an acid sufficient to provide a pH of from about 4 to about 6, such as wherein the pH is about 5, about 5.5, or about 6. In one embodiment, composition has a pH of from about 0 to about 6.5 or less, such as from about 2 to about 6, from about 3 to about 6, such as from about 4 or 4.5 to about 5.5 or less

In one embodiment disclosed herein is a method comprising contacting the lesion and/or an area of the surface adjacent to the lesion with an effective amount of a composition comprising a C1-C10 alcohol and a sufficient amount of an acid to adjust the pH to below 5.5, wherein the lesion is not caused by a virus.

In one embodiment, the present method reduces one or more biomarkers of inflammation, such as IL-2, IL-1β, IL-6, IL-13, IL.-17, IFN-α, TNF-α, IFN-γ and the like. In one embodiment, biomarkers of immune regulation, such as CD3, CD8, CD45 and the like, are increased by treatment according to the present method. In one embodiment, the biomarkers are modulated locally, such as in the skin, the epidermis, the dermis and/or hypodermis. In one embodiment, lesions treated herein are associated with an autoimmune or inflammatory skin disorder, such as, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne or alopecia areata.

In particular embodiments, the lesion comprises a gash, cut, puncture, abrasion, bump, blister, ulcer, or burn on the surface.

In certain embodiments, the lesion is the result of an insect bite or sting.

In particular embodiments, the insect is a mosquito, flea, ant, wasp, bee, hornet, fly, louse, or bedbug.

In additional embodiments, the lesion is the result of an arachnid bite or sting.

In some embodiments, the arachnid is a scorpion, spider, tick, or mite.

In further embodiments, the lesion is the result of an animal bite or sting.

In particular embodiments, the animal bite or sting is the bite or sting of a mammal, a reptile, an amphibian, a cnidarian, or a mollusk.

In particular embodiments, the mammal is a canine, feline, or rodent.

In further embodiments, the reptile is a snake or lizard.

In certain embodiments, the amphibian is a frog, toad, or newt.

In additional embodiments, the cnidarian is a jellyfish, coral, or sea anemone.

In some embodiments, the mollusk is a snail, octopus, or cone shell.

In further embodiments, the lesion is caused by a bacterium or fungus.

In particular embodiments, the lesion is caused by contact with a plant.

In further embodiments, the plant is a Urticacea spp., a Euphorbiaceace spp., a Toxicodendron spp., a blue-green algae, poison ivy, poison oak, poison sumac, a stinging nettle, a dumb cane, a giant hogweed, a blisterbush, a cowhage, an. Indian fig, an upas, a bull nettle, a ciega vista, a blinding tree, a manchineel tree, a sand-box tree, a stinging spurge, a noseburn, a giant stinging tree, a gympie, a nilgri nettle, a wood nettle, a fever nettle, a nettle tree, a cashew nut tree, a rengas, a holigama, a Burma lac tree, a black poison wood, a marking nut tree, a ligas, or a Japanese lacquer tree.

In certain embodiments, the lesion is caused by contact with a chemical irritant.

In additional embodiments, the lesion is a benign lesion, a pre-cancerous lesion, or a cancerous lesion.

In particular embodiments, the lesion treated according to the present method is on a surface selected from the group consisting of: a skin surface, a surface of a mucous membrane surface, and a surface of a nail.

In further embodiments, the surface is selected from the group consisting of: a tracheal surface, a nasal surface, a nasalpharyngeal surface, a pharyngeal surface, a vaginal surface, a gastrointestinal surface, a urothelial surface, and an artificially exposed mucosa or skin surface.

In additional embodiments, the artificially exposed mucosa or skin surface is exposed by a procedure selected from the group consisting of: an incision, a colostomy, an iliostomy, an ileoconduit, a gastrostomy, a ureterostomy, a urethrostomy, a vesciostomy, a bile duct/common bile duct ostomy, and a tracheostomy.

In some embodiments, the composition is formulated as an ointment, paste, drop, tincture, gel, cream, salve, lotion, lip balm, foam, spray, roll-on, or aerosol.

In further embodiments, the C1-C10 alcohol is a straight chain, branched or cyclic alcohol, or is a combination of alcohols. In certain embodiments, the composition comprises a C1-C10 alcohol, such as an alcohol selected from methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, 2-(2 propyl)-5-methyl-cyclohexane-1-ol, or a combination thereof.

In particular embodiments of the disclosed composition, the C1-C10 alcohol comprises a C1 to C3 monohydroxy alcohol or a C1 to C4 diol.

In particular embodiments, the alcohol is selected from the group consisting of methanol, ethanol, 1-propanol, 2-propanol, and combinations thereof.

In certain embodiments, the composition comprises ethanol. In certain such embodiments, the alcohol component is ethanol.

In further embodiments, the diol is selected from the group consisting of 2,3-butanediol, 1,2-butanediol, 1,3-butanediol, 1,4-butanediol, and combinations thereof.

In certain embodiments, the diol is selected from the group consisting of 2,3-butanediol, 1,2-butanediol, 1,3-butanediol, and 1,4-butanediol.

In some embodiments, the composition comprises an organic acid, an inorganic acid, or both. In one embodiment, the acid comprises an organic acid.

In particular embodiments of the compositions disclosed herein comprising an organic acid, the organic acid comprises one or more of glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, or acetic acid.

In certain embodiments of the compositions disclosed herein comprising an organic acid, the organic acid is selected from the group consisting of glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, acetic acid and combinations thereof.

In particular embodiments of compositions disclosed herein, the organic acid comprises glycolic acid.

In certain embodiments of compositions disclosed herein, the organic acid consists essentially of glycolic acid.

In particular embodiments, the acid is an inorganic acid.

In additional embodiments, the inorganic acid comprises hydrochloric acid, boric acid, nitric acid, phosphoric acid, sulfuric acid, hydrobromic acid, hydroiodic acid, hydrosulfuric acid, perchloric acid, or a combination thereof.

In certain embodiments, the composition is contacted to the lesion and/or to a surface adjacent to the lesion a plurality of times.

In further embodiments, the composition is contacted to the lesion and/or adjacent surface tissue a plurality of times in an hour, a day, a week, or a month. In one embodiment, the lesion is contacted with the composition once per day (QD), twice per day (BID), three times per day (TID), or more.

In various embodiments, a method of reducing of decreasing inflammation of a surface of a subject or on the surface lining of an internal organ of a subject is provided comprising contacting the inflamed surface with an effective amount of a composition comprising a C1-C10 alcohol and a sufficient amount of an acid to adjust the pH to below 5.5, wherein the lesion is not caused by a virus.

In some embodiments, the inflammation is the result of a lesion.

In further embodiments, the lesion comprises a gash, cut, puncture, abrasion, bump, blister, ulcer, or burn on the surface.

In additional embodiments, the inflammation is the result of an insect bite or sting.

In additional embodiments, the insect is a mosquito, flea, ant, wasp, bee, hornet, fly, louse, or bedbug.

In further embodiments, the inflammation is the result of an arachnid bite or sting.

In particular embodiments, the arachnid is a scorpion, spider, tick, or mite.

In further embodiments, the inflammation is the result of an animal bite or sting.

In particular embodiments, the animal is a mammal, a reptile, an amphibian, a cnidarian, or a mollusk.

In certain embodiments, the mammal is a canine, feline, or rodent.

In certain embodiments, the reptile is a snake or lizard.

In certain embodiments, the amphibian is a frog, toad, or newt.

In particular embodiments, the cnidarian is a jellyfish, coral, or sea anemone.

In further embodiments, the mollusk is a snail, octopus, or cone shell.

In further embodiments, the inflammation is caused by a bacterial or fungal infection.

In some embodiments, the inflammation is caused by contact with a plant.

In some embodiments, the plant is Urticacea spp., a Euphorbiaceace spp., a Toxicodendron spp., a blue-green algae, poison ivy, poison oak, poison sumac, a stinging nettle, a dumb cane, a giant hogweed, a blisterbush, a cowhage, an Indian fig, an upas, a bull nettle, a ciega vista, a blinding tree, a manchineel tree, a sand-box tree, a stinging spurge, a noseburn, a giant stinging tree, a gympie, a nilgri nettle, a wood nettle, a fever nettle, a nettle tree, a cashew nut tree, a rengas, a holigama, a Burma lac tree, a black poison wood, a marking nut tree, a ligas, or a Japanese lacquer tree.

In certain embodiments, the lesion is caused by contact with a chemical irritant.

In particular embodiments, the inflammation is dermatitis, drug reaction, psoriasis or interstitial cystitis.

In certain embodiments, the inflamed surface is selected from the group consisting of: a skin surface, a surface of a mucous membrane, and a surface of a nail.

In additional embodiments, the inflamed surface is selected from the group consisting of: a tracheal surface, a nasal surface, a nasalpharyngeal surface, a pharyngeal surface, a vaginal surface, a gastrointestinal surface, a urothelial surface, and an artificially exposed mucosa or skin surface.

In some embodiments, the artificially exposed mucosa or skin surface is exposed by a procedure selected from the group consisting of: an incision, a colostomy, an iliostomy, an ileoconduit, a gastrostomy, a ureterostomy, a urethrostomy, a vesciostomy, a bile duct/common bile duct ostomy, and a tracheostomy.

In particular embodiments, the composition is formulated as an ointment, paste, drop, tincture, gel, cream, salve, lotion, lip balm, foam, spray, roll-on, or aerosol.

In particular embodiments for treating an inflamed surface, the C1-C10 alcohol is methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, or 2-(2-propyl)-5-methyl-cyclohexane-1-ol.

In some embodiments, the C1-C10 alcohol is a C1 to C3 monohydroxy alcohol or a C1 to C4 diol.

In further embodiments, the alcohol is selected from the group consisting of methanol, ethanol, 1-propanol, and 2-propanol.

In particular embodiments, the alcohol is ethanol.

In certain embodiments of the present compositions for treating an inflamed surface, the diol is selected from the group consisting of 2,3-butanediol, 1,2-butanediol, 1,3-butanediol, 1,4-butanediol, and combinations thereof.

In particular embodiments, the diol is selected from the group consisting of 2,3-butanediol, 1,2-butanediol, 1,3-butanediol, and 1,4-butanediol.

In some embodiments, the acid is an organic acid.

In additional embodiments for treating an inflamed surface, the organic acid comprises glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, or acetic acid.

In certain embodiments for treating an inflamed surface, the organic acid is selected from glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, acetic acid, and combinations thereof.

In particular embodiments of the method for treating an inflamed surface, the organic acid comprises glycolic acid.

In particular embodiments, the organic acid is glycolic acid.

In further embodiments, the acid is an inorganic acid.

In certain embodiments, the inorganic acid comprises hydrochloric acid, boric acid, nitric acid, phosphoric acid, sulfuric acid, hydrobromic acid, hydroiodic acid, hydrosulfuric acid, or perchloric acid.

In further embodiments, the composition is contacted to the surface a plurality of times.

In further embodiments of the method for treating an inflamed surface, the composition is contacted to the surface a plurality of times in an hour, a day, a week, or a month.

In various embodiments, a method of reducing or decreasing irritation of a surface of a subject is provided comprising contacting the irritated surface with an effective amount of a composition comprising a C1-C10 alcohol and a sufficient amount of an acid to adjust the pH to below 5.5, wherein the lesion is not caused by a virus.

In certain embodiments, the irritation is the result of a lesion.

In particular embodiments, the lesion comprises a gash, cut, puncture, abrasion, bump, blister, ulcer, allergic reaction, or burn on the surface.

In some embodiments, the irritation is the result of an insect bite or sting.

In certain embodiments of the method of reducing irritation, the insect is a mosquito, flea, ant, wasp, bee, hornet, fly, louse, or bedbug.

In further embodiments, the irritation is the result of an arachnid bite or sting.

In additional embodiments, the arachnid is a scorpion, spider, tick, or mite.

In further embodiments, the irritation the result of an animal bite or sting.

In further embodiments, the animal is a mammal, a reptile, an amphibian, a cnidarian, or a mollusk.

In certain embodiments, the reptile is a snake or lizard.

In particular embodiments, the amphibian is a frog, toad, or newt.

In particular embodiments, the cnidarian is a jellyfish, coral, or sea anemone.

In further embodiments, the mollusk is a snail, octopus, or cone shell.

In some embodiments, the irritation is caused by a bacterial or fungal infection.

In particular embodiments, the irritation is caused by a chemical irritant.

In further embodiments, the irritation is caused by contact with a plant.

In additional embodiments, the plant is Urticacea spp., a Euphorbiaceace spp., a Toxicodendron spp., a blue-green algae, poison ivy, poison oak, poison sumac, a stinging nettle, a dumb cane, a giant hogweed, a blisterbush, a cowhage, an Indian fig, an upas, a bull nettle, a ciega vista, a blinding tree, a manchineel tree, a sand-box tree, a stinging spurge, a noseburn, a giant stinging tree, a gympie, a nilgri nettle, a wood nettle, a fever nettle, a nettle tree, a cashew nut tree, a rengas, a holigama, a Burma lac tree, a black poison wood, a marking nut tree, a ligas, or a Japanese lacquer tree.

In additional embodiments, the irritation is caused by inflammation.

In particular embodiments, the inflammation is dermatitis, drug reaction, psoriasis or interstitial cystitis.

In some embodiments, the irritated surface is selected from the group consisting of: a skin surface, a surface of a mucous membrane, and a surface of a nail.

In certain embodiments, the irritated surface is selected from the group consisting of: a tracheal surface, a nasal surface, a nasalpharyngeal surface, a pharyngeal surface, a vaginal surface, a gastrointestinal surface, a urothelial surface, and an artificially exposed mucosa or skin surface.

In further embodiments, the artificially exposed mucosa or skin surface is exposed by a procedure selected from the group consisting of: an incision, a colostomy, an iliostomy, an ileoconduit, a gastrostomy, a ureterostomy, a urethrostomy, a vesciostomy, a bile duct/common bile duct ostomy, and a tracheostomy.

In further embodiments, the composition for reducing irritation is formulated as an ointment, paste, drop, tincture, gel, cream, salve, lotion, lip balm, foam, spray, roll-on, or aerosol.

In particular embodiments, the C1-C10 alcohol is methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, or 2-(2-propyl)-5-methyl-cyclohexane-1-ol.

In further embodiments, the C1-C10 alcohol is a C1 to C3 monohydroxy alcohol or a C1 to C4 diol.

In additional embodiments, the alcohol is selected from the group consisting of methanol, ethanol, 1-propanol, and 2-propanol.

In further embodiments, the alcohol is ethanol.

In certain embodiments, the diol is selected from the group consisting of 2,3-butanediol, 1,2-butanediol, 1,3-butanediol, 1,4-butanediol, and combinations thereof.

In particular embodiments, the diol is selected from the group consisting of 2,3-butanediol, 1,2-butanediol, 1,3-butanediol, and 1,4-butanediol.

In particular embodiments, the acid is an organic acid.

In some embodiments of the composition for reducing irritation, the organic acid comprises one or more of glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, or acetic acid.

In certain embodiments of the composition for reducing irritation, the organic acid is selected from the group consisting of glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, acetic acid, and combinations thereof.

In certain embodiments, the organic acid comprises glycolic acid.

In certain embodiments, the organic acid is glycolic acid.

In certain embodiments, the acid is an inorganic acid.

In some embodiments, the inorganic acid comprises hydrochloric acid, boric acid, nitric acid, phosphoric acid, sulfuric acid, hydrobromic acid, hydroiodic acid, hydrosulfuric acid, or perchloric acid.

In some embodiments, the composition is contacted to the surface a plurality times.

In some embodiments, the composition is contacted to the surface a plurality of times in an hour, a day, a week, or a month.

In various embodiments, a method of reducing or decreasing a bacterial or fungal infection in a subject is provided comprising contacting an infected surface with an effective amount of a composition comprising a C1-C10 alcohol and a sufficient amount of an acid to adjust the pH to below 5.5, wherein the lesion is not caused by a virus.

In particular embodiments, the infection is a bacterial infection.

In additional embodiments, the bacterial infection is caused by a gram negative bacterium.

In further embodiments, the bacterial infection is caused by a gram positive bacterium.

In further embodiments, the bacterial infection is caused by a Mycobacterium spp., a Pneumococcus spp., an Escherichia spp., a Campylobacter spp., a Corynebacterium spp., a Clostridium spp., a Streptococcus spp., a Staphylococcus spp., a Pseudomonas spp., a Shigella spp., a Treponema spp., or a Salmonella spp.

In certain embodiments, the infection is a fungal infection.

In particular embodiments, the fungal infection is caused by an Aspergillis spp., a Blastomyces spp., a Candida spp., a Coccicioides spp., a Cryptococcus spp., dermatophytes, a Tinea spp., a Trichophyton spp., a Microsporum spp., a Fusarium spp., a Histoplasma spp., a Mucoromycotina spp., a Pneumocystis spp., a Sporothrix spp., an Exserophilum spp., or a Cladosporium spp.

In further embodiments, the infected surface is selected from the group consisting of a skin surface, a surface of a mucous membrane, and a surface of a nail.

In particular embodiments, the infected surface is selected from the group consisting of: a tracheal surface, a nasal surface, a nasalpharyngeal surface, a pharyngeal surface, a vaginal surface, a gastrointestinal surface, a urothelial surface, and an artificially exposed mucosa or skin surface.

In some embodiments, the artificially exposed mucosa or skin surface is exposed by a procedure selected from the group consisting of: an incision, a colostomy, an iliostomy, an ileoconduit, a gastrostomy, a ureterostomy, a urethrostomy, a vesciostomy, a bile duct/common bile duct ostomy, and a tracheostomy.

In particular embodiments of the method of reducing or decreasing a bacterial or fungal infection, the composition is formulated as an ointment, paste, drop, tincture, gel, cream, salve, lotion, lip balm, foam, spray, roll-on, or aerosol.

In further embodiments, the C1-C10 alcohol is methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, or 2-(2-propyl)-5-methyl-cyclohexane-1-ol.

In further embodiments, the C1-C10 alcohol is a C1 to C3 monohydroxy alcohol or a C1 to C4 diol.

In particular embodiments, the alcohol is selected from the group consisting of methanol, ethanol, 1-propanol, and 2-propanol.

In particular embodiments, the alcohol is ethanol.

In particular embodiments, the diol is selected from the group consisting of 2,3-butanediol, 1,2-butanediol, 1,3-butanediol, 1,4-butanediol, and combinations thereof.

In particular embodiments, the diol is selected from the group consisting of 2,3-butanediol, 1,2-butanediol, 1,3-butanediol, and 1,4-butanediol.

In certain embodiments, the acid is an organic acid.

In additional embodiments of the method of reducing infection, the organic acid comprises one or more of glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, or acetic acid.

In certain embodiments, the organic acid is selected from the group consisting of glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, acetic acid, and combinations thereof.

In further embodiments, the acid is an inorganic acid.

In some embodiments, the inorganic acid comprises hydrochloric acid, boric acid, nitric acid, phosphoric acid, sulfuric acid, hydrobromic acid, hydroiodic acid, hydrosulfuric acid, or perchloric acid.

In particular embodiments, the composition is contacted to the surface a plurality times.

In further embodiments of the method for reducing infection, the composition is contacted to the surface a plurality of times in an hour, a day, a week, or a month.

In various embodiments disclosed herein, a method of reducing or decreasing pain in a subject wherein the pain is caused by a lesion, inflammation, irritation, or bacterial or fungal infection of a surface of a subject is provided, comprising contacting the painful surface with an effective amount of a composition comprising a C1-C10 alcohol and a sufficient amount of an acid to adjust the pH to below 7, such as less than about 6.5, below 6 or below about 5.5. In one embodiment, the method comprises treating a lesion with an effective amount of a composition comprising a C1-C10 alcohol and a sufficient amount of an acid to adjust the pH to composition has a pH of from about 0 to about 6.5 or less, such as from about 2 to about 6, from about 3 to about 6, such as from about 4 or 4.5 to about 5.5 or less, wherein the lesion is not caused by a virus.

In certain embodiments, the pain is the result of a lesion of the surface.

In further embodiments, the lesion comprises a gash, cut, puncture, abrasion, bump, blister, ulcer, allergic reaction, or burn on the surface.

In particular embodiments, the pain is the result of inflammation of the surface.

In some embodiments, the pain is the result of irritation of the surface.

In additional embodiments, the pain is the result of an insect bite or sting.

In further embodiments, the insect is a mosquito, flea, ant, wasp, bee, hornet, fly, louse, or bedbug.

In particular embodiments, the pain is the result of an arachnid bite or sting.

In particular embodiments, the arachnid is a scorpion, spider, tick, or mite.

In some embodiments, the pain is the result of an animal bite or sting.

In certain embodiments, the animal is a mammal, a reptile, an amphibian, a cnidarian, or a mollusk.

In further embodiments, the reptile is a snake or lizard.

In certain embodiments, the amphibian is a frog, toad, or newt.

In further embodiments, the cnidarian is a jellyfish, coral, or sea anemone.

In additional embodiments, the mollusk is a snail, octopus, or cone shell.

In additional embodiments, the pain is caused by a bacterial or fungal infection.

In certain embodiments, the pain is caused by a chemical irritant.

In particular embodiments, the pain is caused by contact with a plant.

In particular embodiments, the plant is Urticacea spp., a Euphorbiaceace spp., a Toxicodendron spp., a blue-green algae, poison ivy, poison oak, poison sumac, a stinging nettle, a dumb cane, a giant hogweed, a blisterbush, a cowhage, an Indian fig, an upas, a bull nettle, a ciega vista, a blinding tree, a manchineel tree, a sand-box tree, a stinging spurge, a nosebum, a giant stinging tree, a gympie, a nilgri nettle, a wood nettle, a fever nettle, a nettle tree, a cashew nut tree, a rengas, a holigarna, a Burma lac tree, a black poison wood, a marking nut tree, a ligas, or a Japanese lacquer tree.

In particular embodiments, the pain is caused by or is coincident with an infection.

In particular embodiments, the infection is a bacterial infection.

In further embodiments, the bacterial infection is caused by a gram negative bacterium.

In certain embodiments, the bacterial infection is caused by a gram positive bacterium.

In some embodiments, the bacterial infection is caused by a Mycobacterium spp., a Pneumococcus spp., an Escherichia spp., a Campylobacter spp., a Corynebacterium spp., a Clostridium spp., a Streptococcus spp., a Staphylococcus spp., a Pseudomonas spp., a Shigella spp., a Treponema spp., or a Salmonella spp.

In some embodiments, the infection is a fungal infection.

In certain embodiments, the fungal infection is caused by an Aspergillis spp., a Blastomyces spp., a Candida spp., a Coccicioides spp., a Cryptococcus spp., dermatophytes, a Tinea spp., a Trichophyton spp., a Microsporum spp., a Fusarium spp., a Histoplasma spp., a Mucoromycotina spp., a Pneumocystis spp., a Sporothrix spp., an Exserophilum spp., or a Cladosporium spp.

In some embodiments, the painful surface is selected from the group consisting of: a skin surface, a surface of a mucous membrane, and a surface of a nail.

In particular embodiments, the surface is selected from the group consisting of: a tracheal surface, a nasal surface, a nasalpharyngeal surface, a pharyngeal surface, a vaginal surface, a gastrointestinal surface, a urothelial surface, and an artificially exposed mucosa or skin surface.

In additional embodiments, the artificially exposed mucosal surface is exposed by a procedure selected from the group consisting of: an incision, a colostomy, an iliostomy, an ileoconduit, a gastrostomy, a ureterostomy, a urethrostomy, a vesciostomy, a bile duct/common bile duct ostomy, and a tracheostomy.

In further embodiments, the composition for treating the painful surface is formulated as an ointment, paste, drop, tincture, gel, cream, salve, lotion, lip balm, foam, spray, roll-on, or aerosol.

In particular embodiments, the C1-C10 alcohol is methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, or 2-(2-propyl)-5-methyl-cyclohexane-1-ol.

In particular embodiments, the C1-C10 alcohol is a C1 to C3 monohydroxy alcohol or a C1 to C4 diol.

In certain embodiments, the alcohol is selected from the group consisting of methanol, ethanol, 1-propanol, and 2-propanol.

In certain embodiments, the alcohol is ethanol.

In certain embodiments, the diol is selected from the group consisting of 2,3-butanediol, 1,2-butanediol, 1,3-butanediol, 1,4-butanediol, and combinations thereof.

In certain embodiments, the diol is selected from the group consisting of 2,3-butanediol, 1,2-butanediol, 1,3-butanediol, and 1,4-butanediol.

In some embodiments, the acid is an organic acid.

In further embodiments, the organic acid comprises one or more of glycolic acid, lactic acid, squaric acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, or acetic acid.

In further embodiments, the organic acid is glycolic acid.

In additional embodiments, the acid is an inorganic acid.

In some embodiments, the inorganic acid comprises hydrochloric acid, boric acid, nitric acid, phosphoric acid, sulfuric acid, hydrobromic acid, hydroiodic acid, hydrosulfuric acid, or perchloric acid.

In particular embodiments, the composition is contacted to the surface a plurality times.

In particular embodiments, the composition is contacted to the surface a plurality of times in an hour, a day, a week, or a month.

In one aspect, the composition for treating a lesion as described herein consists essentially of an alcohol and an acid.

In certain embodiments, the composition consists of an alcohol and an acid as the active components.

In further embodiments, the pH of the composition ranges from pH 2.0 to about pH 5.5.

In certain embodiments, the pH of the composition ranges is about pH 3.5 or lower.

In further embodiments, the pH of the composition is about pH 3.0 or lower.

In additional embodiments, the pH of the composition is about pH 2.5 or lower.

In some embodiments, the alcohol concentration in the composition ranges from about 0.2% by volume up to about 40% by volume.

In further embodiments, the alcohol concentration in the composition ranges from about 0.5% by volume up to about 20% by volume.

In further embodiments, the alcohol concentration in the composition ranges from about 1% by volume up to about 15% by volume.

In particular embodiments, the composition comprises: about 0.2% to about 15% ethanol or from about 1% or about 2% to about 12% ethanol; and about 0.1% w/v to about 5% w/v glycolic acid, or about 0.2% w/v to about 4% w/v glycolic acid, or about 0.2% w/v to about 4% w/v glycolic acid, or about 0.3% w/v to about 3% w/v glycolic acid, or about 0.4% w/v to about 2% w/v glycolic acid, or about 0.5% w/v to about 1% w/v glycolic acid.

In certain embodiments, the composition comprises about 10% ethanol; and about 0.6% w/v glycolic acid.

In certain embodiments, the composition comprises a pharmaceutically acceptable excipient.

In further embodiments, the composition excludes an amphoteric or pseudoamphoteric compound.

In particular embodiments, the composition excludes an amino acid and/or a peptide.

In some embodiments, the composition excludes a dipeptide and/or a tripeptide.

In additional embodiments, the composition excludes an imidazoline amphoteric and/or a lecithin amphoteric.

In certain embodiments, the composition excludes one or more compounds selected from the group consisting of cocoamphoglycine, cocoamphoproprionate, cocoamphopropylsulfonate, phosphatidyl ethanolamine, phosphatidyl serine, and sphingomyelin.

In particular embodiments, the composition is formulated as a unit dosage formulation.

### BRIEF DESCRIPTION OF THE FIGURES

FIG. 1A illustrates the protocol for an in vivo wound healing study performed as described herein.

FIG. 1B provides digital images of a vehicle-treated wound vs. a wound treated according to the present methods over a 12 day time course.

FIG. 1C charts the percentage of open wound area versus time for the vehicle-treated group vs. the group treated according to the present methods.

FIG. 2 illustrates wound healing efficiency represented by percentage of healing wounds (Y-axis) plotted as a function of time (X-axis).

FIG. 3A provides digital images of a vehicle-treated wound vs. a wound treated according to the present methods over a 12 day time course.

FIG. 3B charts the percentage of open wound area versus time for the vehicle-treated group vs. the group treated according to the present method.

FIG. 4 illustrates wound healing efficiency represented by percentage of healing wounds (Y-axis) plotted as a function of time (X-axis).

FIG. 5 provides representative digital images of CD8 stained control grafts (panels a and b) and grafts treated with the acidic composition according to the present methods (panels c and d), demonstrating that the present composition blocks the recruitment of CD8+ cytotoxic T-cells and demonstrates the anti-inflammatory effect of treatment as described herein.

FIG. 6 provides a bar graph showing the % CD8 staining per field in the 10 vehicle-treated and 10 groups treated with the present acidic composition (labeled “drug”).

FIG. 7 provides a bar graph showing the distribution of the number of grafts present in 0-25%, 25-50% and 50-75% staining category in control and treated group.

FIG. 8 provides representative digital images of CD4 stained grafts from two controls (panels a and b) and two grafts treated with the present composition (panels c and d) at ×2.5 magnification.

FIG. 9 provides a bar graph showing the % CD CD4 positive cells in the 6 control grafts and 7 grafts treated with the present acidic composition.

FIG. 10 provides representative digital images of FOXP3 stained grafts from two controls (panels a and b) and two grafts treated with the present composition (panels c and d).

FIG. 11 provides a bar graph showing the % FOXP3 positive cells in 5 control and 5 treated grafts.

FIG. 12 provides images of two skin burn samples showing the distance between the migratory tongue in the burn samples treated with PBS only (negative control)(E indicates the epidermis and D indicates the dermis).

FIG. 13 provides images of skin burn samples showing the distance between the migratory tongue in the burn samples treated with a composition disclosed herein (GE: Glycolic acid 0.6%+10% Ethanol (pH 2.5)).

FIG. 14 provides images of skin burn samples showing the distance between the migratory tongue in the burn samples treated with a 0.6% glycolic acid solution (G: Glycolic acid 0.6% (pH 2.5)).

FIG. 15 provides images of skin burn samples showing the distance between the migratory tongue in the burn samples treated with human serum added in the medium (Positive Control).

FIG. 16 provides a bar graph reporting the distance between the migratory tongue in skin burn samples exposed to different treatments (GE: Glycolic acid 0.6%+10% Ethanol (pH 2.5); G: Glycolic acid 0.6% (pH 2.5); C: Cannabinoid (0.1 mg/ml)).

FIG. 17 provides a bar graph reporting the statistical analysis results (two tailed Unpaired t-test) of an Agar diffusion assay of the ability of GE: Glycolic acid 0.6%+10% Ethanol (pH2.5) and G: Glycolic acid 0.6% (pH 2.5) compositions to inhibit the growth of E. coli and S. aureus.

### DETAILED DESCRIPTION

### A. Overview

The present disclosure provides compositions suitable for the prevention, treatment, and/or amelioration of at least one symptom of a condition affecting a surface of a subject's body. Treatment of conditions caused by viruses may be excluded from all embodiments contemplated herein. Illustrative examples of these conditions include, but are not limited to lesions, inflammation, irritation, infection, or pain affecting a surface of a subject's body, e.g., integumentary system, respiratory system, digestive system, reproductive system, urinary system, and mucous membranes. In one embodiment the compositions and methods for their use described herein relate to dermatological conditions, including, without limitation, those caused by acute injuries, inflammatory conditions and autoimmune disorders. The compositions contemplated herein display unexpected synergy in decreasing the healing time of various types of conditions and restoring the affected area to a normal healthy state.

In various embodiments, methods of preventing, treating, or ameliorating at least one symptom of a lesion are contemplated. In a particular embodiment, a method comprises contacting the lesion or an area proximal to or affected by the lesion with an effective amount of an acidic composition comprising a C1-C10 alcohol, such as a composition comprising a sufficient amount of an acid to adjust the pH to below 7, such as below about 6.5, such as about 6 or below about 5.5.

In particular embodiments, methods of preventing, treating, or ameliorating at least one symptom of inflammation of a body surface are contemplated. In a particular embodiment, a method comprises contacting the inflamed surface or an area proximal to or affected by the inflammation with an effective amount of an acidic composition comprising a C1-C10 alcohol, such as a composition comprising a sufficient amount of an acid to adjust the pH to below pH 6 or below about pH 5.5.

In certain embodiments, methods of preventing, treating, or ameliorating at least one symptom of irritation of a body surface using the present compositions are contemplated. In a particular embodiment, a method comprises contacting the irritated surface or an area proximal to or affected by the irritation with an effective amount of an acidic composition comprising a C1-C10 alcohol, such as a composition comprising a sufficient amount of an acid to adjust the pH to below 6 or below about 5.5.

In further embodiments, methods of preventing, treating, or ameliorating at least one symptom of infection of a body surface using the present compositions are contemplated. In a particular embodiment, a method comprises contacting a bacterially or fungally infected surface or an area proximal to or affected by the infection with an effective amount of an acidic composition comprising a C1-C10 alcohol, such as a composition comprising a sufficient amount of an acid to adjust the pH to below 6 or below about pH 5.5.

In additional embodiments, methods of preventing or treating pain in a subject using the present compositions are contemplated. In particular embodiments, the pain may be caused by a lesion, inflammation, irritation, or bacterial or fungal infection. In a particular embodiment, a method comprises contacting the painful surface or an area proximal to or affected by the pain with an effective amount of an acidic composition comprising a C1-C10 alcohol, such as a composition comprising a sufficient amount of an acid to adjust the pH to below 6 or below about pH 5.5.

In various embodiments, a composition comprising an alcohol and an acid, at a pH below about 6, or below about 5.5 is contemplated for the prevention, treatment, and/or amelioration of at least one symptom of conditions including, but are not limited to lesions, inflammation, irritation, infection, or pain affecting a surface of a subject's body. While alcohols and acids do not display potent healing properties when used individually, the present inventors surprisingly and unexpectedly discovered that a combination of one or more short chain alcohols and one or more acids to adjust the pH to below 5.5 displays very potent healing properties against a wide range of conditions affecting the integumentary system, respiratory system, digestive system, reproductive system, urinary system, and mucous membranes, wherein the conditions are not caused by viruses.

### B. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred embodiments of compositions, methods and materials are described herein. For the purposes of the present invention, the following terms are defined below.

The articles “a,” “an,” and “the” are used herein to refer to one or to more than one (i.e., to at least one) of the grammatical object of the article. By way of example, “an element” means one element or more than one element.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements. In one embodiment, a composition consists essentially of an organic acid, e.g., glycolic acid, and a C1-C10 alcohol, wherein the composition is at an acidic pH, that is, a pH of less than 7. In certain embodiments, the C1-C10 alcohol comprises a C1-C4 alcohol; e.g., ethanol, and the composition is at a pH of less than 5.5.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “surface,” “surface area,” or “body surface” refer to the surface of an organ of the integumentary system, respiratory system, digestive system, reproductive system, urinary system, or a mucous or skin membrane. In particular embodiments, the term “surface” includes an external surface of an organ. In particular embodiments, the term “surface” includes an internal surface of an organ.

The “integumentary system” refers to an organ system that includes the skin, hair (hair, feathers, scales), nails, and exocrine glands.

“Skin” refers to the largest organ of the body. The skin provides a protective barrier that protects us from microbes and the elements, helps regulate body temperature, and permits the sensations of touch, heat, and cold. The skin has three layers: the epidermis, the outermost layer of skin, provides a waterproof barrier and creates our skin tone; the dermis, beneath the epidermis, contains tough connective tissue, hair follicles, and sweat glands; and the deeper subcutaneous tissue (hypodermis) made of fat and connective tissue. In particular embodiments, “skin” refers to one or more or all three of the layers of the skin. In certain embodiments, “skin” refers to a lesion of the epidermis and dermis. In other embodiments, “skin” refers to the epidermis.

As used herein, the terms “mucous membrane” or “mucosa” refer to a membrane lining bodily cavities and canals that lead to the outside, chiefly the respiratory system, gastrointestinal system, reproductive system, and urinary system. Mucous membranes line many tracts and structures of the body, including the mouth, Eustachian tube and sinuses; tear ducts, nose, eyelids, windpipe and lungs, trachea, bronchus, bronchioles, and alveoli, stomach and intestines, pancreatic ducts, biliary ducts, Fallopian tubes, the ureters, urethra, and urinary bladder. The membranes vary in structure, but they all have a surface layer of epithelial or transepithelial cells over a deeper layer of connective tissue.

“Nails” refer to accessory organs of the skin made of sheets of hardened keratinocytes that found on the distal ends of the fingers and toes. There are 3 main parts of a nail: the root, body, and free edge. The nail root is the portion of the nail found under the surface of the skin. The nail body is the visible external portion of the nail. The free edge is the distal end portion of the nail that has grown beyond the end of the finger or toe. In particular embodiments, “nail” refers to one or more or all three of the layers of the skin. In certain embodiments, “nail” refers to the root, body, and/or free edge.

The terms “treating,” “treatment,” and the like are used herein to generally mean obtaining a desired pharmacologic and/or physiologic effect. The effect may be prophylactic in terms of completely or partially preventing a condition affecting a body surface and/or may be therapeutic in terms of a partial or complete cure of the condition. “Treatment” as used herein covers any treatment in a subject, and includes: preventing the condition from occurring in a subject which may be predisposed but has not yet been diagnosed; inhibiting the condition, i.e., arresting its development; or providing relief, i.e., causing regression. The therapeutic compositions may be administered before, during or after the onset of the condition.

As used herein, the phrase “ameliorating at least one symptom of” refers to decreasing one or more symptoms of a condition for which the subject is being treated. In particular embodiments, the condition being treated is a lesion, inflammation, irritation, or infection associated with a body surface, wherein the one or more symptoms ameliorated include, but are not limited to, pain, inflammation, infection, redness, fever, immune reaction, edema, fibrosis, scabbing, oozing, chemical reaction, allergic reaction, and toxic reaction. In particular embodiments, the condition being treated is a lesion wherein the one or more symptoms ameliorated include pain, inflammation, irritation, and/or infection.

A “lesion” refers to a disruption in the integrity of a surface. In particular embodiments, the term lesion refers to a gash, a cut, a puncture, an abrasion, a bump, a blister, an ulcer, allergic reaction, or a burn on the surface. In other embodiments, a lesion is contemplated to include or exclude a gash, a cut, a puncture, an abrasion, a bump, a blister, an ulcer, or a burn on the surface.

As used herein, “prevent,” and similar words such as “prevented,” “preventing” etc., indicate an approach for preventing, inhibiting, or reducing the likelihood of the occurrence or recurrence of a condition. It also refers to delaying the onset or recurrence of a condition or delaying the occurrence or recurrence of the symptoms of a condition. As used herein, “prevention” and similar words also include reducing the intensity, effect, symptoms and/or burden of a condition prior to onset or recurrence of the condition.

As used herein, the term “amount” refers to “an amount effective” or “an effective amount” of composition sufficient to achieve a beneficial or desired prophylactic or therapeutic result, including clinical results. In one embodiment, an effect amount refers to the amount of alcohol and acid in a composition sufficient to prevent, ameliorate one symptom of, or treat a condition affecting a body surface or mucous membranes.

A “prophylactically effective amount” refers to a composition comprising an amount of alcohol and acid to achieve the desired prophylactic result. Typically but not necessarily, since a prophylactic dose is used in subjects prior to or at an earlier stage of a condition, the prophylactically effective amount is less than the therapeutically effective amount.

A “therapeutically effective amount” refers to an amount of a composition that may vary according to factors such as the disease state, age, sex, and weight of the individual, and the agent to elicit a desired response in the individual. A therapeutically effective amount is also one in which any toxic or detrimental effects of the active agents, e.g., alcohol and acid, are outweighed by the therapeutically beneficial effects. The term “therapeutically effective amount” includes an amount that is effective to “treat” a subject (e.g., a patient).

As used herein, the terms “conditions sufficient,” or “under conditions sufficient,” refer to the conditions for treating the subject, with the compositions contemplated herein. In one embodiment, “conditions sufficient” include administering a sufficient amount, e.g., a composition comprising an effective amount of alcohol and acid to a subject in need thereof.

As used herein, the terms “promoting,” “enhancing,” “stimulating,” or “increasing” generally refer to the ability of compositions contemplated herein to produce or cause a greater physiological response (i.e., measurable downstream effect), as compared to the response caused by either vehicle or a control molecule/composition. One such measurable physiological response includes, without limitation, increased healing of a lesion or increased relief from inflammation, irritation, infection, or pain of a body surface compared to normal, untreated, or control-treated subjects. The physiological response may be increased by at least 5%, 10%, 20%, 30%, 40%, 50%, 60%, 70%, 80%, 90%, 100%, 125%, 150%, 175%, 200%, or greater compared to the response measured in normal, untreated, or control-treated subjects. An “increased” or “enhanced” response or property is typically “statistically significant” and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) that produced by normal, untreated, or control-treated subjects.

As used herein, the terms “decrease” or “lower,” or “lessen,” or “reduce,” or “abate” refers generally to the ability of compositions contemplated to produce or cause a lesser physiological response (i.e., downstream effects), as compared to the response caused by either vehicle or a control molecule/composition, e.g., decreased severity of the lesion, decreased inflammation, decreased irritation, decreased infection, lesion, inflammation, irritation, or infection associated with a body surface, decrease in one or more symptoms including, but not limited to, inflammation, infection, redness, fever, immune reaction, edema, fibrosis, reduced scabbing, reduced oozing, or decreased pain. A “decrease” or “reduced” response is typically a “statistically significant” response, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response produced by normal, untreated, or control-treated subject.

A “subject,” “subject in need of treatment,” “subject in need thereof,” “individual,” or “patient” as used herein, includes any animal that exhibits a symptom of a condition that can be treated with compositions contemplated herein. In particular embodiments, the condition relates to a lesion of a body surface, inflammation of a body surface, irritation of a body surface, infection of a body surface, and or pain associated with a body surface. Suitable subjects include laboratory animals (such as mouse, rat, rabbit, or guinea pig), farm animals (such as horses, cows, sheep, pigs), and domestic animals or pets (such as a cat or dog). In particular embodiments, the subject is a mammal. In certain embodiments, the subject is a non-human primate and, in preferred embodiments, the subject is a human.

An “alcohol” refers to include primary, secondary, and tertiary alcohols.

The term “acid” refers to a substance that acts as a proton donor (Brønsted-Lowry definition). In certain embodiments, an acid is any substance that ionizes to give the H⁺, or hydrogen, ion, or more accurately, hydronium ions (H₃O⁺), when dissolved in water (Arrhenius definition), to acids that are aqueous solutions or that can be dissolved in water.

### C. Compositions

In various embodiments, compositions comprise one or more alcohols and one or more acids such that the composition is acidic. In certain embodiments, the acid composition comprises a C1-C10 alcohol, such as a composition comprising a sufficient amount of an acid to adjust the pH to below 7, such as less than about 6.5, below 6 or below about 5.5. In certain embodiments, the composition comprises a sufficient amount of an acid to have a pH of less than 5.5. Typically the acidic compositions employed herein have a pH of from about 0 to 7, such as from about 0 to about 6.5, from 0 to about 6 or from about 2 to about 6.5, such as from about 3 to 7 or from 3.5 to about 6, such as about 4, about 4.5, about 5, about 5.5, or about 6. The compositions described herein are suitable for the prevention, treatment, and/or amelioration of at least one symptom of a lesion, or a surface of a subject's body suffering from or affected by inflammation, irritation, infection, or pain. The combinations of acids and alcohols described herein display unexpected synergy in decreasing the healing time of various types of conditions and restoring the affected area to a normal healthy state. Additionally this synergy permits the alcohol and/or acid to be used at substantially lower concentrations while providing increased antiviral activity thereby permitting such formulations to be used on sensitive lesions and/or tissues.

In various embodiments, a composition comprises an alcohol and an acid, optionally comprising one or more additional active components. In particular embodiments, a composition comprises a C1-C10 alcohol, or a C1-C8 alcohol, or a C1-C6 alcohol, or a C1-C4 alcohol; an acid; and a pharmaceutically acceptable carrier; wherein the pH of the composition is acidic, such as a pH of less than 7, such as less than 6.5, less than 6, less than 5.5, or less than about pH 5.4, or less than about pH 4.5, or less than about pH 4.0, or less than about pH 3.5, or less than about pH 3.0, or less than about pH 2.5. In certain embodiments, the composition comprises a C1-C10 alcohol, or a C1-C8 alcohol, or a C1-C6 alcohol, or a C1-C4 alcohol; an acid; and a pharmaceutically acceptable carrier; wherein the pH of the composition is acidic, such as a pH of from about 4 to about 7, such as from about 4.5 to about 6.5, from about 4 to about 6, from about 5 to about 6.5 or from about 5 to about 5.5.

In particular embodiments, a composition comprises a C1-C10 alcohol, or a C1-C8 alcohol, or a C1-C6 alcohol, or a C1-C4 alcohol; an organic acid selected from the group consisting of glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, or acetic acid; and a pharmaceutically acceptable carrier; wherein the pH of the composition is less than 5.5, or less than about pH 5.4, or less than about pH 4.5, or less than about pH 4.0, or less than about pH 3.5, or less than about pH 3.0, or less than about pH 2.5. In certain embodiments, the composition comprises a C1-C10 alcohol, or a C1-C8 alcohol, or a C1-C6 alcohol, or a C1-C4 alcohol; an organic acid; and a pharmaceutically acceptable carrier; wherein the pH of the composition is acidic, such as a pH of from about 4 to about 7, such as from about 4.5 to about 6.5, from about 4 to about 6, from about 5 to about 6.5 or from about 5 to about 5.5.

In particular embodiments, a composition comprises a C1-C10 alcohol, or a C1-C8 alcohol, or a C1-C6 alcohol, or a C1-C4 alcohol; an inorganic acid selected from the group consisting of hydrochloric acid, boric acid, nitric acid, phosphoric acid, sulfuric acid, hydrobromic acid, hydroiodic acid, hydrosulfuric acid, or perchloric acid; and a pharmaceutically acceptable carrier; wherein the pH of the composition is less than 5.5, or less than about pH 5.4, or less than about pH 4.5, or less than about pH 4.0, or less than about pH 3.5, or less than about pH 3.0, or less than about pH 2.5. In certain embodiments, the composition comprises a C1-C10 alcohol, or a C1-C8 alcohol, or a C1-C6 alcohol, or a C1-C4 alcohol; an inorganic acid; and a pharmaceutically acceptable carrier; wherein the pH of the composition is acidic, such as a pH of from about 4 to about 7, such as from about 4.5 to about 6.5, from about 4 to about 6, from about 5 to about 6.5 or from about 5 to about 5.5.

In another embodiment, a composition comprises a C1-C10 alcohol, or a C1-C8 alcohol, or a C1-C6 alcohol, or a C1-C4 alcohol; a beta hydroxy acid or an omega hydroxy acid; and a pharmaceutically acceptable carrier; wherein the pH of the composition is less than 5.5, or less than about pH 5.4, or less than about pH 4.5, or less than about pH 4.0, or less than about pH 3.5, or less than about pH 3.0, or less than about pH 2.5. In certain embodiments, the composition comprises a C1-C10 alcohol, or a C1-C8 alcohol, or a C1-C6 alcohol, or a C1-C4 alcohol; a beta hydroxy acid, a omega hydroxy acid, or both; and a pharmaceutically acceptable carrier; wherein the pH of the composition is acidic, such as a pH of from about 4 to about 7, such as from about 4.5 to about 6.5, from about 4 to about 6, from about 5 to about 6.5 or from about 5 to about 5.5.

In certain embodiments, a composition comprises one or more alcohols selected from the group consisting of methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, or 2-(2-propyl)-5-methyl-cyclohexane-1-ol; an acid; and a pharmaceutically acceptable carrier; wherein the pH of the composition is acidic, such as less than about 7, less than about 6.5, less than about 6, less than 5.5, or less than about pH 5.4, or less than about pH 4.5, or less than about pH 4.0, or less than about pH 3.5, or less than about pH 3.0, or less than about pH 2.5.

In particular embodiments, a composition comprises one or more alcohols selected from the group consisting of methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, or 2-(2-propyl)-5-methyl-cyclohexane-1-ol; one or more organic acids selected from the group glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, or acetic acid and a pharmaceutically acceptable carrier; wherein the pH of the composition is less than 5.5, or less than about pH 5.4, or less than about pH 4.5, or less than about pH 4.0, or less than about pH 3.5, or less than about pH 3.0, or less than about pH 2.5.

In particular embodiments, a composition comprises one or more alcohols selected from the group consisting of methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, or 2-(2-propyl)-5-methyl-cyclohexane-1-ol; one or more inorganic acids selected from the group consisting of hydrochloric acid, boric acid, nitric acid, phosphoric acid, sulfuric acid, hydrobromic acid, hydroiodic acid, hydrosulfuric acid, or perchloric acid; and a pharmaceutically acceptable carrier; wherein the pH of the composition is less than 5.5, or less than about pH 5.4, or less than about pH 4.5, or less than about pH 4.0, or less than about pH 3.5, or less than about pH 3.0, or less than about pH 2.5.

In various embodiments, a composition comprises one or more alcohols selected from the group consisting of butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, and 2-(2-propyl)-5-methyl-cyclohexane-1-ol; an acid; and a pharmaceutically acceptable carrier; where the pH of the composition is about pH 5.5 or lower.

Illustrative examples of alcohol/acid combinations include those combinations disclosed in U.S. Provisional Application No. 62/214,038” filed Sep. 3, 2015, and entitled “TOPICAL ANTIVIRAL COMPOSITIONS,” the disclosure of which, including the alcohol/acid combinations disclosed therein, is incorporated herein by reference in its entirety.

In particular embodiments, the C1-C10 alcohol is a C1 to C3 monohydroxy alcohol or a C1 to C4 diol.

In certain embodiments, the alcohol is selected from the group consisting of methanol, ethanol, 1-propanol, and 2-propanol.

In other embodiments, the diol is selected from the group consisting of 2,3-butanediol, 1,2-butanediol, 1,3-butanediol, and 1,4-butanediol.

In one embodiment, a composition comprises ethanol and glycolic acid.

In particular embodiments, a composition contemplated herein, further comprises one or more agents selected from the group consisting of an antioxidant phenolic compound, a tannin, a dicarboxylic acid or derivative thereof, mandelic acid, an extract from the plant Prunella vulgaris, and vitamin C.

Illustrative examples of antioxidant phenolic compounds suitable for incorporation in the compositions contemplated herein include, but are not limited to, phenol, and resorcinol.

Illustrative examples of dicarboxylic acids suitable for incorporation in the compositions contemplated herein include, but are not limited to, squaric acid, succinic acid, and tartaric acid.

In particular embodiments, certain compounds are excluded from the compositions contemplated herein. In certain embodiments, any of the compositions contemplated herein excludes one or more of an amphoteric or pseudoamphoteric compound; an amino acid and/or a peptide; a dipeptide and/or a tripeptide; an imidazoline amphoteric and/or a lecithin amphoteric; a cocoamphoglycine, cocoamphoproprionate, cocoamphopropylsulfonate, phosphatidyl ethanolamine, phosphatidyl serine, or sphingomyelin.

In certain embodiments, the composition is not a mouthwash and/or is not suitable for use as a mouthwash.

In various embodiments, a composition comprises one or more alcohols at a concentration of about 0.2% by volume up to about 40% by volume. In certain embodiments, a composition comprises one or more alcohols at a concentration of about 0.5% by volume up to about 30% or up to about 20% by volume. In certain embodiments, a composition comprises one or more alcohols at a concentration of about 1% by volume up to about 15% by volume or about 2% by volume up to about 13% by volume or about 2% up to about 10% by volume.

In one embodiment, a composition comprises one or more alcohols at a concentration of about 5%, about 6%, about 7%, about 8%, or about 9%, or about 10%, or about 11%, or about 12%, or about 13%, or about 14%, or about 15%, or about 16%, or about 17%, or about 18%, or about 19%, or about 20%, or about 21%, or about 22%, or about 23%, or about 24%, or about 25%, or about 26%, or about 27%, or about 28%, or about 29%, or about 30%, or about 31%, or about 32%, or about 33%, or about 34%, or about 35%, or about 36%, or about 38%, or about 38%, or about 39%, or about 40% by volume.

In certain embodiments, a composition comprises one or more acids at a concentration of about 0.1% to about 50%, or about 0.1% to about 40%, or about 0.1% to about 30%, or about 0.1% to about 20%, or about 0.1% to about 15%, or about 0.1% to about 10%, or about 0.1% to about 5%, or about 0.2% to about 4%, or about 0.3% to about 3%, or about 0.4% to about 2% by weight. In certain embodiments, a composition comprises one or more acids at a concentration of about 0.1, or about 0.2%, or about 0.3%, or about 0.4%, or about 0.5%, or about 0.6%, or about 0.7%, or about 0.8%, or about 0.9%, or about 1.0%, or about 1.1%, or about 1.2%, or about 1.3%, or about 1.4%, or about 1.5%,or about 1.6%, or about 1.7%, or about 1.8%, or about 1.9%, or about 2.0% by weight.

In certain embodiments, a composition comprises one or more acids at a concentration of about 0.1%, or about or 0.2%, or about 0.3%, or about 0.4%, or about 0.5%, or about 0.6%, or about 0.7%, or about 0.8%, or about 0.9%, or about 1.0%, or about 1.1%, or about 1.2%, or about 1.3%, or about 1.4%, or about 1.5%, or about 1.6%, or about 1.7%, or about 1.8.%, or about 1.9%, or about 2.0%, or about 2.1%, or about 2.2%, or about 2.3%, or about 2.4%, or about 2.5%, or about 3%, or about 3.5%, or about 4%, or about 4.5%, or about 5%, or about 5.5%, or about 6%, or about 6.5%, or about 7%, or about 7.5%, or about 8%, or about 8.5%, or about 89%, or about 9.5%, or about 10%, or about 11%, or about 12%, or about 13%, or about 14%, or about 15%, or about 16%, or about 17%, or about 18%, or about 19%, or about 20%, or about 21%, or about 22%, or about 23, 24%, or about 25%, or about 26%, or about 27%, or about 28%, or about 29%, or about 30%, or about 31%, or about 32%, or about 33%, or about 34%, or about 35%, or about 36%, or about 37%, or about 38%, or about 39%, or about 40%, by weight.

In particular embodiments, a composition comprises about 0.2% to about 15% C1-C10 alcohol or from about 1% or about 2% to about 12% C1-C10 alcohol; and about 0.1% w/v to about 5% w/v acid, or about 0.2% w/v to about 4% w/v acid, or about 0.2% w/v to about 4% w/v acid, or about 0.3% w/v to about 3% w/v acid, or about 0.4% w/v to about 2% w/v acid, or about 0.5% w/v to about 1% w/v acid.

In one embodiment, a composition comprises about 10% ethanol; and about 0.6% w/v glycolic acid.

In particular embodiments, a composition comprises one or more acids at a concentration sufficient to adjust the pH of the composition to less than about pH 5.5 or lower, or to about pH 4.5 or lower, or to about pH 4.0 or lower, or to about pH 3.5 or lower, or to about pH 3.0 or lower, or to about pH 2.5 or lower.

In particular embodiments, a composition comprises one or more acids at a concentration sufficient to adjust the pH of the composition to about pH 5.5 or less, or about pH 4.5 or less, or about pH 4 or less, or about pH 3.5 or less, or about pH 3 or less, or about pH 2.5 or less, or about pH 2.

In certain embodiments, a composition comprises one or more acids at a concentration sufficient to adjust the pH of the composition to about pH 1.5 or pH 2 up to about pH 5.5, or up to about pH 4.5, or up to about pH 4, or up to about pH 3.5, or up to about pH 3, or up to about pH 2.5.

Compositions (i.e., medicaments) contemplated herein include, but are not limited to pharmaceutical compositions. A “pharmaceutical composition” refers to a composition formulated with one or more pharmaceutically acceptable carriers, diluents or excipients generally accepted in the art for the delivery of therapeutic agents to a subject either alone, or in combination with one or more other modalities of therapy. In particular embodiments, pharmaceutical compositions comprise a C1-C10 alcohol, or a C1-C8 alcohol, or a C1-C6 alcohol, or a C1-C4 alcohol; an acid; and a pharmaceutically acceptable carrier, diluent or excipient; wherein the pH of the composition is less than 5.5, or less than about pH 5.4, or less than about pH 4.5, or less than about pH 4.0, or less than about pH 3.5, or less than about pH 3.0, or less than about pH 2.5. It will also be understood that, if desired, the compositions described herein may be administered in combination with other agents as well, such as, e.g., nucleic acids, proteins, small molecules, or pharmaceutically-active agents, adjunct therapies, etc. so long as the desired therapeutic effect is achieved. In certain embodiments, there is virtually no limit to other components that may also be included in the compositions, provided that the additional components do not adversely affect the desired effect to be achieved.

In various embodiments the compositions described herein can be employed in mixture with conventional excipients, i.e., pharmaceutically acceptable organic or inorganic carrier substances which do not react with the acid or the alcohol in the composition.

“Pharmaceutically acceptable” refers to those compounds, agents, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals.

Illustrative examples of pharmaceutically acceptable carriers include, but are not limited to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions, and the like, and any other compatible substances employed in pharmaceutical compositions which do not deleteriously react with the acid or the alcohol in the composition.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In particular embodiments, a composition contemplated herein is formulated so as to allow the active ingredients contained therein to be bioavailable upon administration of the composition to a subject. In one embodiment, pharmaceutical compositions can be prepared by combining a C1-C10 alcohol and an acid with an appropriate pharmaceutically acceptable carrier, diluent or excipient, and may be formulated into preparations in solid, semi-solid, liquid, gels, and microspheres.

Pharmaceutical compositions may comprise a pharmaceutically-acceptable carrier, diluent, or excipient is well-known to those of skill in the art, as is the development of suitable dosing and treatment regimens for using the particular compositions contemplated herein in a variety of treatment regimens, including, e.g., topical, enteral, and parenteral administration.

In certain embodiments, the pharmaceutical compositions disclosed herein may be delivered via enteral administration to a subject. Illustrative examples of enteral administration include, but are not limited to delivering the compositions orally, buccally, or intragastrically. Compositions for enteral administration can be formulated in a tablet (coated or uncoated), capsule (hard or soft), microsphere, emulsion, powder, granule, crystal, suspension, syrup or elixir. Supplementary active compounds (e.g., preservatives, antibacterial, antiviral and antifungal agents) can also be incorporated into the compositions. A liquid compositions can also be used for enteral administration. The carrier can be selected from various oils including petroleum, animal, vegetable or synthetic, for example, peanut oil, soybean oil, mineral oil, sesame oil. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, and water.

In particular embodiments, the pharmaceutical compositions disclosed herein may be administered parenterally. Illustrative examples of parenteral administration include, but are not limited to delivering the compositions subcutaneously, intravenously, intramuscularly, intra-arterially, intrathecally, intraparenchymally, intracisternally, intraventricularlly, intraurethrally, intrastemally, intracranially, intrasynovially, or even intraperitoneally as described, for example, in U.S. Pat. Nos. 5,543,158; 5,641,515 and 5,399,363 (each specifically incorporated herein by reference in its entirety). Suitable devices for parenteral administration include needle (including microneedle) injectors, needle-free injectors, and infusion techniques.

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. Prolonged absorption of the injectable compositions can be brought about by the use in the compositions of agents delaying absorption, for example, aluminum monostearate and gelatin.

Sterile injectable solutions can be prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Pharmaceutical compositions contemplated herein may also be administered topically. Illustrative examples of topical administration include, but are not limited to delivering the compositions (intra)dermally or transdermally to the skin or nails, or vaginally, intranasally, or by inhalation to mucous membranes. Other illustrative examples of topical administration include delivery by electroporation, iontophoresis, phonophoresis, sonophoresis, and microneedle or needle-free injection for example using the systems sold under the trademarks POWDERJECT™, and BIOJECTrm.

Illustrative examples of typical compositions for topical administration include, but are not limited to, gels, hydrogels, lotions, solutions, creams, ointments, dusting powders, dressings, foams, films, skin patches, wafers, implants, sponges, fibers, bandages, liposomes, and microemulsions. Illustrative examples of typical carriers include, but are not limited to, alcohol, water, mineral oil, liquid petrolatum, white petrolatum, glycerin, polyethylene glycol, and propylene glycol. Topical compositions may further comprise penetration enhancers including, but not limited to, see also e.g., Finnin and Morgan: J. Pharm. Sci. 88(10): 955-958, (1999).

In various embodiments, a composition is suitable for topical administration to a subject (e.g., to a human or to a non-human mammal). Methods that are customary in industrial pharmaceutics can be employed to produce topical compositions. For this, the active compounds (e.g., the alcohol and acid) are processed, preferably together with a pharmaceutically acceptable carrier particularly a carrier suitable for application on the skin and/or mucosa.

It will be recognized that the various active components (e.g., alcohol(s), acids(s), additional components, etc.) are water soluble and, in certain embodiments, can simply be admixed in an aqueous solution and/or as a tincture. In certain embodiments, the composition is for topical application, that is, a liquid comprising an alcohol and an acid.

Other suitable forms include, but are not limited to, semi-solid or solid forms comprising a carrier indigenous to topical application and typically having a dynamic viscosity preferably greater than that of water, provided that the carrier does not deleteriously react with the acid, the alcohol, or when present the additional component(s) in the composition. Suitable compositions include, but are not limited to, lip balms, suspensions, emulsions, creams, ointments, powders, liniments, salves and the like. In particular embodiments, the compositions are sterilized or mixed with auxiliary agents, e.g., preservatives, stabilizers, wetting agents, buffers or salts for influencing osmotic pressure and the like.

In certain embodiments vehicles for semi-solid or solid forms topical preparations include, but are not limited to ointment bases, e.g., polyethylene glycol-1000 (PEG-1000); creams, e.g., HEB cream; and gels, e.g., K-Y gel; as well as petroleum jelly and the like. Topical compositions contemplated herein may also contain emollients, perfumes, and/or pigments to enhance their acceptability for various uses, provided that the additives do not deleteriously react with the acid or the alcohol in the composition.

In certain embodiments, pharmaceutical compositions are formulated as sprayable aerosol preparations wherein the composition, optionally in combination with a solid or liquid inert carrier material, is packaged in a squeeze bottle or in admixture with a pressurized volatile, normally gaseous propellant. Suitable propellants include, but are not limited to chlorofluorocarbons, hydrocarbons, and hydrocarbon ethers. The aerosol or spray compositions may further comprise contain solvents, buffers, surfactants, perfumes, and/or antioxidants.

Pharmaceutical compositions may also be formulated as gels, ointments, creams of the mixed-phase or amphiphilic emulsion systems (oil/water-water/oil mixed phase), and also liposomes and transfersomes, glycerosomes, or plasters, ointments and creams, for application to the body surface.

Additional topically applicable forms that can be produced include pastes, foams, gels, powders, creams, ointments, and the like. As consistency-imparting bases, the pastes frequently comprise hydrophobic and hydrophilic auxiliary substances. In certain embodiments, hydrophobic auxiliary substances are provide having a high solids content in order to increase their dispersity and flowability and glidability, and also to prevent agglomerates. In certain embodiments the powders or topically applicable powders can also contain, for example, starch types, such as wheat starch or rice starch, flame-disperse silicon dioxide or silicaceous earths, which also serve as diluents.

Pharmaceutical compositions may also be formulated as emulsions, creams, ointments, foam tablets or suppositories for application to the genitals, vagina, or rectum. Rectal capsules can also be produced on the basis of gelatin or other carrier substances. Examples of suitable suppository bases are hydrogenated fats, such as Witepsol®, Massa Estarium®, Novata®, coconut butter, glycerol/gelatin compositions, glycerol/saponaceous gels and polyethylene glycols.

Controlled release compositions may be made by formulating the compositions with biocompatible polymers, viscosity agents, gels, paints, foams, xerogels, microparticles, hydrogels, nanocapsules, and thermoreversible gels, or combinations thereof. In particular embodiments, the polymer or gels are biodegradable. Release properties are often controlled by the particular combination of polymers or gels used to formulate the composition. These methods are well known in the art.

Exemplary polymers suitable for use in the compositions contemplated herein include, but are not limited to polyamides, polycarbonates, polyalkylenes (polyethylene glycol (PEG)), polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-cocaprolactone), polysaccharides, proteins, polyhyaluronic acids, polycyanoacrylates, and blends, mixtures, or copolymers thereof.

In particular embodiments, the polymer is a ABA-type or BAB-type triblock copolymers or mixtures thereof, wherein the A-blocks are relatively hydrophobic and comprise biodegradable polyesters or poly(orthoester), and the B-blocks are relatively hydrophilic and comprise polyethylene glycol (PEG). The biodegradable, hydrophobic A polymer block comprises a polyester or poly(ortho ester), in which the polyester is synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxyhexanoic acid, γ-butyrolactone, γ-hydroxybutyric acid, δ-valerolactone, δ-hydroxyvaleric acid, hydroxybutyric acids, malic acid, and copolymers thereof.

Exemplary viscosity agents suitable for use in the compositions contemplated herein include, but are not limited to, hydroxypropyl methylcellulose, hydroxyethyl cellulose, polyvinylpyrrolidone, carboxymethyl cellulose, polyvinyl alcohol, sodium chondroitin sulfate, sodium hyaluronate, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, or polyvinylpyrrolidone (PVP: povidone).

Gelling agents suitable for use in the compositions contemplated herein include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, glycerin-based gels, glycerin-derived compounds, conjugated, or crosslinked gels, matrices, hydrogels, and polymers, as well as gelatins and their derivatives, and various native and synthetic hydrogel and hydrogel-derived compounds, and any combinations or mixtures thereof.

Pharmaceutical compositions formulated as gels can be present either as water-based hydrogels or as hydrophobic organogels, for example, based on mixtures of low molecular weight and high molecular weight paraffin hydrocarbons and vaseline. The hydrophilic organogels can, for example, be prepared on the basis of high molecular weight polyethylene glycol. These gelatinous forms can be washed off. However, the organogels which are preferred are the hydrophobic organogels. Particular preference is given to hydrophobic auxiliary substances and additives such as petroleum jelly, wax, oleyl alcohol, propylene glycol monostearate and propylene glycol monopalmitostearate. Furthermore, it is possible to add dyes, for example yellow and/or red iron oxide and/or titanium dioxide, for the purpose of color adjustment.

In certain embodiments, emulsifying agents can also be included in the compositions. Illustrative examples of emulsifying agents that can be used include anionic, cationic or neutral surfactants, for example alkali soaps, metal soaps, amine soaps, sulfurized and sulfonated compounds, invert soaps, high fatty alcohols, partial fatty acid esters of sorbitan and polyoxyethylene sorbitan, e.g., lanette types, wool fat, lanolin and other synthetic products for producing oil/water and/or water/oil emulsions. Other examples of suitable auxiliary substances are ionic or anionic detergents, such as Triton X-100, Tween, sodium deoxycholate, and also polyols, such as polyethylene glycol or glycerol, sugars, for example sucrose or glucose, lipopolysaccharides, zwitterionic compounds, such as amino acids, such as glycine or, in particular, taurine or betaine, or lipids.

Vaseline, natural or synthetic waxes, fatty acids, fatty alcohols, fatty acid esters, for example as monoglycerides, diglycerides or triglycerides, paraffin or vegetable oils, hydrogenated castor oil or coconut oil, lard, synthetic fats, for example based on caprylic acid, capric acid, lauric acid and stearic acid, such as Softisan®, or triglyceride mixtures, such as Miglyol®, can be used as lipids in the form of fatty and/or oily and/or waxy components for producing the ointments, creams or emulsions.

In certain embodiments, a composition comprises one or more compounds to increase its stability including, but not limited to, preservatives, such as methyl benzoate or propyl benzoate (parabene), sorbic acid; proteins, such as bovine, human or synthetic serum albumin; protease inhibitors, such as aprotinin; and/or c hydrophobic esters, such as isopropyl laureate, isopropyl myristate, isopropyl palmitate, isopropyl stearate, ethyl myristate, propyl myristate, butyl myristate and/or ethyl oleate, in particular isopropyl myristate. In this aspect, the term “hydrophobic” is understood as referring to compounds whose solubility in water is at most approx. 0.2 mg/ml, in particular at most approx. 0.1 mg/ml.

The pharmaceutical compositions may be formulated to be immediate and/or sustained release. Sustained release includes delayed, modified, pulsed, controlled, targeted and programmed release. Thus, the compositions may be formulated as a suspension or as a solid, semi-solid, or thixotropic liquid for administration as an sustained release composition. Examples of such compositions include without limitation, drug-coated stents and semi-solids and suspensions comprising drug-loaded poly(DL-lactic-co-glycolic)acid (PGLA), poly(DL-lactide-co-glycolide) (PLG) or poly(lactide) (PLA) lamellar vesicles or microparticles, hydrogels (Hoffman AS: Ann. N.Y. Acad. Sci. 944: 62-73 (2001)), poly-amino acid nanoparticles systems, sold under the trademark MEDUSA® developed by Flamel Technologies Inc., non-aqueous gel systems sold under the trademark ATRIGEL® developed by Atrix, Inc., and Sucrose Acetate Isobutyrate Extended Release formulations sold under the trademark SABER® developed by Durect Corporation, and lipid-based systems developed by SkyePharma and sold under the trademark DEPOFOAM®.

Sustained release devices capable of delivering desired doses of the pharmaceutical compositions over extended periods of time are known in the art. For example, U.S. Pat. Nos. 5,034,229; 5,557,318; 5,110,596; 5,728,396; 5,985,305; 6,113,938; 6,156,331; 6,375,978; and 6,395,292; teach osmotically-driven devices capable of delivering an active agent composition, such as a solution or a suspension, at a desired rate over an extended period of time (i.e., a period ranging from more than one week up to one year or more). Other exemplary sustained release devices include regulator-type pumps that provide constant flow, adjustable flow, or programmable flow of beneficial agent compositions, which are available from Medtronic including the Intrathecal pumps sold under the trademark SYNCHROMED INFUSION SYSTEM®, the Johnson and Johnson systems sold under the trademark CODMAN® division pumps, and INSET® technologies pumps. Further examples of devices are described in U.S. Pat. Nos. 6,283,949; 5,976,109; 5,836,935; and 5,511,355.

Pharmaceutical compositions may also include anti-contamination agents for the prevention of microorganism contamination. Anti-contamination agents may include but are not limited to antibacterial and antifungal agents, for example, paraben, chlorobutanol, phenol sorbic acid, antibiotics, and the like.

Pharmaceutical compositions may also be sterilized by, for example, by filtration through a bacteria-retaining filter, or by incorporating sterilizing agents in the form of sterile solid compositions which can be dissolved in sterile water, or some other sterile medium immediately before use or composition.

Pharmaceutical compositions may also be endotoxin free. As used herein, the term “endotoxin free” refers to compositions that contain at most trace amounts (i.e., amounts having no adverse physiological effects to a subject) of endotoxin, and preferably undetectable amounts of endotoxin. By “substantially free of endotoxin” is meant that there is less endotoxin per dose of cells than is allowed by the FDA for a biologic, which is a total endotoxin of 5 EU/kg body weight per day, which for an average 70 kg person is 350 EU per total dose of cells. In one embodiment, the term “endotoxin free” refers to a composition that is at least 95%, at least 96%, at least 97%, at least 98%, at least 99%, or 100% endotoxin free. Endotoxins are toxins associated with certain bacteria, typically gram-negative bacteria, although endotoxins may be found in gram-positive bacteria, such as Listeria monocytogenes. The most prevalent endotoxins are lipopolysaccharides (LPS) or lipooligosaccharides (LOS) found in the outer membrane of various Gram-negative bacteria, and which represent a central pathogenic feature in the ability of these bacteria to cause disease. Small amounts of endotoxin in humans can produce fever, a lowering of the blood pressure, and activation of inflammation and coagulation, among other adverse physiological effects. Therefore, it is often desirable to remove most or all traces of endotoxin from drug product containers, because even small amounts may cause adverse effects in humans.

Pharmaceutical compositions are administered in a manner compatible with the dosage form and in such amount as is therapeutically effective to result in an improvement or remediation of the symptoms. The compositions are easily administered in a variety of dosage forms such as ingestible solutions, drug release capsules, gel ointments and the like. Some variation in dosage can occur depending on the condition of the subject being treated. The person responsible for administration can, in any event, determine the appropriate dose for the individual subject. Moreover, for human administration, preparations meet sterility, general safety and purity standards as required by FDA Office of Biologics standards.

One embodiment of the present invention contemplates the use of any of the pharmaceutical compositions contemplated herein to make a medicament for preventing, treating or ameliorating at least one symptom of condition affecting a body surface, e.g., a lesion, inflammation, irritation, infection, or pain. Medicaments can be formulated based on the physical characteristics of the patient/subject needing treatment, and can be formulated as single or multiple dosage forms based on the stage of the condition. Medicaments contemplated herein can be packaged in a suitable pharmaceutical package with appropriate labels for the distribution to hospitals and clinics wherein the label is for the indication of treating a condition of a body surface as described herein in a subject. Medicaments can be packaged as a single dosage units or multiple dosage units. Instructions for the dosage and administration of the pharmaceutical compositions contemplated herein can be included with the pharmaceutical packages and kits.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

Additional methods of preparing pharmaceutical compositions are known to the skilled artisan, for example, as described in the Physicians' Desk Reference, 62nd edition. Oradell, NJ: Medical Economics Co., 2008; Goodman & Gilman's The Pharmacological Basis of Therapeutics, Eleventh Edition. McGraw-Hill, 2005; Remington: The Science and Practice of Pharmacy, 20th Edition. Baltimore, MD: Lippincott Williams & Wilkins, 2000; and The Merck Index, Fourteenth Edition. Whitehouse Station, NJ: Merck Research Laboratories, 2006; each of which is hereby incorporated by reference in relevant parts.

In one embodiment, the pharmaceutical compositions additionally provide an immediate release of one or more pharmaceutically active ingredients (i.e., C1-C10 alcohols and an acid) from the composition, or within 1 minute, or within 5 minutes, or within 10 minutes, or within 15 minutes, or within 30 minutes, or within 60 minutes or within 90 minutes.

In another embodiment, a therapeutically effective amount of at least one pharmaceutically active ingredient is released from the composition immediately, or within 1 minute, or within 5 minutes, or within 10 minutes, or within 15 minutes, or within 30 minutes, or within 60 minutes or within 90 minutes.

In yet another embodiment, a pharmaceutical composition provides an extended release. In certain embodiments, diffusion of at least one pharmaceutically active ingredient from the pharmaceutical composition occurs for a time period exceeding 5 minutes, 15 minutes, 30 minutes, 1 hour, 4 hours, 6 hours, 12 hours, 18 hours, 1 day, 2 days, 3 days, 4 days, 5 days, 6 days, 7 days, 10 days, 12 days, 14 days, 18 days, 21 days, 25 days, or 30 days.

In further embodiments, a pharmaceutical composition provides both an immediate release and an extended release. In particular embodiments, the pharmaceutical composition contains a 0.25:1 ratio, a 0.5:1 ratio, a 1:1 ratio, a 1:2 ratio, a 1:3, a 1:4 ratio, a 1:5 ratio, a 1:7 ratio, a 1:10 ratio, a 1:15 ratio, or a 1:20 ratio of immediate release and extended release compositions. In a further embodiment, the composition provides an immediate release of two or more pharmaceutically active ingredients and an extended release of two or more of the same of different pharmaceutically active ingredients.

In additional embodiments, the composition provides a 0.25:1 ratio, a 0.5:1 ratio, a 1:1 ratio, a 1:2 ratio, a 1:3, a 1:4 ratio, a 1:5 ratio, a 1:7 ratio, a 1:10 ratio, a 1:15 ratio, or a 1:20 ratio of immediate release and extended release compositions of one or more pharmaceutically active ingredients.

In various embodiments, the development of suitable dosing and treatment regimens for using the particular compositions contemplated herein in a variety of treatment regimens including, e.g., topical, enteral, and parenteral administration is well known in the art.

In some embodiments, a composition comprising one or more C1-C10 alcohols and acids is administered at least once during a treatment cycle. In particular embodiments, a composition is administered to the subject over one or more treatment cycles. A treatment cycle can be at least 1, at least 2, at least 3, at least 4, at least 5, at least 6, at least 7, at least 8, at least 9, at least 10, at least 12, at least 18, or at least 24 hours, or at least 2, at least 3, at least 4, at least 5, at least 6, at least 7, at least 14, at least 21, at least 28, at least 48, or at least 96 days or more.

In some embodiments, a composition is administered one or more times for at least one day, at least two days, at least three days, at least four days, at least five days, at least six days, at least seven days, at least eight days, at least nine days, at least ten days, at least eleven days, at least twelve days, at least 13 days, at least 14 days, at least 21 days, or all 28 days of a 28 day treatment cycle. In particular embodiments, a composition is administered to a subject once a day. In other particular embodiments, a composition is administered twice a day. In certain embodiments, a composition is administered more than twice a day.

The number of times a composition is administered to a subject in need thereof depends on the nature and severity of the injury or condition, and the subject's response to the composition. In some embodiments, a composition disclosed herein is administered once to a subject in need thereof with a mild acute condition. In some embodiments, a composition disclosed herein is administered more than once to a subject in need thereof with a moderate or severe acute condition. In the case wherein the subject's condition does not improve, the composition may be administered chronically, that is, for an extended period of time in order to ameliorate or otherwise control or limit the symptoms of the subject's injury or condition.

### D. Methods of Treatment

The pharmaceutical compositions contemplated herein can be used to prevent, treat, or ameliorate at least one symptom associated with a condition affecting a surface of a subject, wherein the condition is not caused by a virus. In preferred embodiments, the surface is a component of the integumentary system, including but not limited to, skin or nails, or a mucous membrane. A “condition affecting a surface” or a “condition associated with a surface” refers to a superficial injury such as a lesion or infection or inflammation, irritation or a pain on an area of the surface. The condition may be at the site of a lesion, or adjacent or near the lesion. The pharmaceutical compositions contemplated herein comprise one or more alcohols and acids that display unexpected synergy in decreasing the healing time of various types of conditions and restoring the affected area to a normal healthy state.

In various embodiments, methods of treating a surface are contemplated, the methods comprising contacting an area of the surface affected by a condition with an effective amount of a composition comprising a C1-C10 alcohol and an acid, wherein the pH of the composition is less than 5.5, or less than about pH 5.4, or less than about pH 4.5, or less than about pH 4.0, or less than about pH 3.5, or less than about pH 3.0, or less than about pH 2.5.

In one embodiment, the condition is a lesion. In particular embodiments, the present invention contemplates, in part, a method of reducing or decreasing the severity or size of a lesion on a body surface comprising contacting the lesion with an effective amount of a composition comprising a C1-C10 alcohol and an acid, wherein the pH of the composition is less than 5.5, or less than about pH 5.4, or less than about pH 4.5, or less than about pH 4.0, or less than about pH 3.5, or less than about pH 3.0, or less than about pH 2.5, wherein the lesion is not caused by a virus.

In some embodiments, the condition is a lesion that may be treated with the compositions contemplated herein including, but are not limited to, a gash, a cut, a puncture, an abrasion, a bump, a blister, an ulcer, allergic reaction, or a burn on the surface.

In one embodiment, the condition is inflammation. In particular embodiments, the present invention contemplates, in part, a method of reducing or decreasing inflammation of a body surface comprising contacting the inflamed surface with an effective amount of a composition comprising a C1-C10 alcohol and an acid, wherein the pH of the composition is less than 5.5, or less than about pH 5.4, or less than about pH 4.5, or less than about pH 4.0, or less than about pH 3.5, or less than about pH 3.0, or less than about pH 2.5, wherein the inflammation is not caused by a virus.

Illustrative examples of causes of inflammation that are preventable or treatable with the compositions contemplated herein include, but are not limited to inflammation caused by, lesions, irritation, infection, skin reactions, allergic reactions, asthma, lung diseases or responses, acute inflammatory diseases, chronic inflammation, immune related diseases, wound healing, interstitial cystitis, and arthritis.

In one embodiment, the condition is irritation. In particular embodiments, the present invention contemplates, in part, a method of reducing or decreasing irritation of a body surface comprising contacting the irritated surface with an effective amount of a composition comprising a C1-C10 alcohol and an acid, wherein the pH of the composition is less than 5.5, or less than about pH 5.4, or less than about pH 4.5, or less than about pH 4.0, or less than about pH 3.5, or less than about pH 3.0, or less than about pH 2.5, wherein the irritation is not caused by a virus.

Illustrative examples of causes of irritation that are preventable or treatable with the compositions contemplated herein include, but are not limited to irritation caused by, lesions, inflammation, infection, skin reactions, allergic reactions, reactions to chemicals, heat rash, razor burn, and wound healing.

In one embodiment, the condition is a bacterial or fungal infection. In particular embodiments, the present invention contemplates, in part, a method of reducing or decreasing a bacterial or fungal infection comprising contacting an infected surface with an effective amount of a composition comprising a C1-C10 alcohol and an acid, wherein the pH of the composition is less than 5.5, or less than about pH 5.4, or less than about pH 4.5, or less than about pH 4.0, or less than about pH 3.5, or less than about pH 3.0, or less than about pH 2.5, wherein the infection is not caused by a virus.

In certain embodiments, the condition is caused by a bacterial or fungal infection. In one embodiment, the bacterial infection is caused by a gram negative bacterium. In another embodiment, the bacterial infection is caused by a gram positive bacterium.

In a particular embodiment, the compositions are applied to a surface to prevent, treat, or ameliorate pain of an area of a surface. In particular embodiments, the present invention contemplates, in part a method of reducing or decreasing a painful condition in a subject wherein the pain is caused by a lesion, inflammation, irritation, or bacterial or fungal infection of a body surface comprising contacting the painful area with an effective amount of a composition comprising a C1-C10 alcohol and an acid, wherein the pH of the composition is less than 7, such as less than 6.5, such as 6 or less, less than 5.5, or less than about pH 5.4, or less than about pH 4.5, or less than about pH 4.0, or less than about pH 3.5, or less than about pH 3.0, or less than about pH 2.5, wherein the pain is not caused by a virus.

In particular embodiments, compositions contemplated herein are contacted to an affected surface area, or area proximal, close to, or near the affected surface area. In various embodiments, the surface area is selected from the group consisting of: a skin surface, a surface of a mucous membrane surface, and a surface of a nail. In certain embodiments, the surface is a tracheal surface, a nasal surface, a nasalpharyngeal surface, a pharyngeal surface, a vaginal surface, a gastrointestinal surface, urothelial surface, or an artificially exposed mucosa or skin surface.

Illustrative examples of procedures that artificially expose mucosal surfaces include, but are not limited to an incision, a colostomy, an iliostomy, an ileoconduit, a gastrostomy, a ureterostomy, a urethrostomy, a vesciostomy, a bile duct/common bile duct ostomy, or a tracheostomy.

In particular embodiments, the condition is the product of animal bite or sting. In some embodiments, the compositions contemplated herein may be used to treat animal bites and stings from animals including, but not limited to: insects, including but not limited to mosquitoes, fleas, ants, wasps, bees, hornets, flies, lice, and bedbugs; arachnids, including but not limited to scorpions, spiders, ticks, and mites; reptiles, including but not limited to snakes and lizards; amphibians, including but not limited to frogs, toads, and newts; cnidarians, including but not limited to corals, jellyfish and sea anemones; mollusks, including but not limited to octopus, snails, cone shells; and mammals, including without limitation, rodents, felines, canines, caprines, ovines, equines, bovines, ursines, and other animals.

In some embodiments, the compositions contemplated herein are suitable for treating bacterial infections caused by a Mycobacterium spp., a Pneumococcus spp., an Escherichia spp., a Campylobacter spp., a Corynebacterium spp., a Clostridium spp., a Streptococcus spp., a Staphylococcus spp., a Pseudomonas spp., a Shigella spp., a Treponema spp., or a Salmonella spp. or another species of pathogenic bacteria.

In one embodiment, the compositions contemplated herein are suitable for treating bacterial infections caused by Streptococcus mutans, such as is present in dental caries and/or periodontal disease (gum disease).

In some embodiments, the compositions contemplated herein are suitable for treating fungal infections caused by yeasts.

In some embodiments, the compositions contemplated herein are suitable for treating fungal infections caused by an Aspergillis spp., a Blastomyces spp., a Candida spp., a Coccicioides spp., a Cryptococcus spp., dermatophytes, a Tinea spp., a Trichophyton spp., a Microsporum spp., a Fusarium spp., a Histoplasma spp., a Mucoromycotina spp., a Pneumocystis spp., a Sporothrix spp., an Exserophilum spp., or a Cladosporium spp. or another species of pathogenic fungus.

In various embodiments, the condition is caused by contact with a plant. In some embodiments, the compositions contemplated herein are suitable for treating conditions caused by contact with a plant. In some embodiments, the compositions contemplated herein are suitable for treating conditions caused by a plant including, but not limited to a Urticacea spp., a Euphorbiaceace spp., a Toxicodendron spp., blue-green algae, poison ivy, poison oak, poison sumac, stinging nettle, dumb cane, giant hogweed, blisterbush, cowhage, Indian fig, upas, bull nettle, ciega vista, blinding tree, manchineel tree, sand-box tree, stinging spurge, noseburn, giant stinging tree, gympie, nilgri nettle, wood nettle, fever nettle, nettle tree, cashew nut tree, rengas, holigarna, Burma lac tree, black poison wood, marking nut tree, ligas, or Japanese lacquer tree.

In particular embodiments, the condition is caused by contact with a chemical irritant. In some embodiments, the compositions contemplated herein are suitable for treating conditions caused by a chemical irritant including, but not limited to plant chemicals, household cleaning agents, including but not limited to ammonia, trisodium phosphate, isopropanol, bleach, lye, and sulfuric acid; active agents in laundry detergents including but not limited to, surfactants, enzymes, bleach, and fragrances; active agents in sunscreens, including but not limited to zinc oxide, titanium oxide, and para-aminobenzoic acid; active agents in bug repellents including, but not limited to N-diethyl-meta-toluamide (DEET); use of hair removal products; cosmetics; soaps; and dyes or chemical additives in clothing fabrics.

In various embodiments, the condition is a benign lesion, pre-cancerous lesion, a cancerous lesion or a lesion resulting from a pre-cancerous condition or cancer. The lesions may be caused by: a squamous cell carcinoma of the skin, head and neck, thyroid, esophagus, lung, penis, prostate, vagina, and cervix, and bladder; oropharyngeal cancers, basal cell carcinoma of the skin, and melanoma.

In various embodiments, methods of using the compositions contemplated herein as surgical or post-surgical washes are provided comprising washing an surface or area that is being surgically manipulated and/or that has been subject to surgery with an effective amount of a composition comprising a C1-C10 alcohol and an acid, wherein the pH of the composition is less than 5.5, or less than about pH 5.4, or less than about pH 4.5, or less than about pH 4.0, or less than about pH 3.5, or less than about pH 3.0, or less than about pH 2.5.

In various embodiments, methods of treating a surface are contemplated, the methods comprising contacting an area of the surface affected by a condition with an effective amount of a composition comprising a C1-C10 alcohol and an acid, wherein the pH of the composition is less than 5.5, or less than about pH 5.4, or less than about pH 4.5, or less than about pH 4.0, or less than about pH 3.5, or less than about pH 3.0, or less than about pH 2.5; and further providing an adjunct therapy including, but not limited to, an analgesic, a non-steroidal anti-inflammatory (NTHE), a local anesthetic agent, a steroid, an anti-cancer therapy, and an anti-infective agent.

In one embodiment, a subject is treated with a composition comprising a C1-C10 alcohol and an acid, and an analgesic. The analgesic can be an opioid or non-opioid analgesic.

Illustrative examples of opioid analgesics include, but are not limited to hydrocodone, oxycodone, morphine, diamorphine, codeine, pethidine, alfentanil, buprenorphine, butorphanol, codeine, dezocine, fentanyl, hydromorphone, levomethadyl acetate, levorphanol, meperidine, methadone, morphine sulfate, nalbuphine, oxycodone, oxymorphone, pentazocine, propoxyphene, remifentanil, sufentanil, tramadol or a pharmaceutically acceptable salt thereof.

Illustrative examples of non-opioid analgesics include, but are not limited to aspirin, amoxiprin, benorilate, choline magnesium salicylate, diflunisal, faislamine, methyl salicylate, magnesium salicylate, diclofenac, aceclofenac, acemetacin, bromfenac, etodolac, indometacin, nabumetone, sulindac, tolmetin, ibuprofen, carprofen, fenbuprofen, flubiprofen, ketaprofen, ketorolac, loxoprofen, naproxen, suprofen, mefenamic acid, meclofenamic acid, piroxicam, lomoxicam, meloxicam, tenoxicam, phenylbutazone, azapropazone, metamizole, oxyphenbutazone, sulfinprazone, tramadol, or a pharmaceutically acceptable salt thereof.

In one embodiment, a subject is treated with a composition comprising a C1-C10 alcohol and an acid; and a non-steroidal anti-inflammatory.

Illustrative examples of NTHEs include, but are not limited to: a COX-1 inhibitor; a COX-2 inhibitor, including but not limited to diaryl-substituted furanones (e.g., Rofecoxib); diaryl-substituted pyrazoles (e.g., Celecoxib); indole acetic acids (e.g., Etodolac); and sulfonanilides (e.g., Nimesulide); a salicylic acid; a salicylic acid derivative, including, but not limited to choline magnesium trisalicylate, salsalate, diflunisal, salicylsalicylic acid, sulfasalazine, olsalazine, esters of salicylic acid with a carboxylic acid, esters of salicylic acid with a dicarboxylic acid, esters of salicylic acid with a fatty acid, esters of salicylic acid with a hydroxyl fatty acid, esters of salicylic acid with an essential fatty acid, esters of salicylic acid with a polycarboxylic acid; para-aminophenol; an indole or an indole-acetic acid derivative (e.g., indomethacin, sulindac, etodolac); an aryl acetic acids (e.g., tolmetin, diclofenac, ketorolac); an arylpropionic acid or derivative including, but not limited to ibuprofen, naproxen, flubiprofen, ketoprofen, fenoprofen, oxaprozin; anthranilic acids or an anthranilic acid derivative, also termed “fenamates” (e.g., mefenamic acid, meclofenamic acid); enolic acids, enolic acid salts, enolic acid esters, amides, anhydrides and derivatives thereof including, but not limited to oxicams (piroxicam, tenoxicam) and pyrazolidinediones (phenylbutazone, oxyphenthratrazone); and an alkanone (e.g., nabumetone).

In one embodiment, a subject is treated with a composition comprising a C1-C10 alcohol and an acid; and a local anesthetic agent.

Illustrative examples of local anesthetic agents include, but are not limited to: benzocaine, lidocaine, bupivacaine, chlorprocaine, dibucaine, etidocaine, mepivacaine, tetracaine, dyclonine, hexylcaine, procaine, cocaine, ketamine, pramoxine, phenol, any pharmaceutically acceptable salts thereof and mixtures of such anesthetic agents.

In one embodiment, a subject is treated with a composition comprising a C1-C10 alcohol and an acid, and a steroid.

Illustrative examples of steroids include, but are not limited to: a corticosteroid, including but not limited to, hydrocortisone, hydroxyltriamcinolone, alpha-methyl dexamethasone, dexamethasone-phosphate, beclomethsone dipropionate, clobetasol valemate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortmate, mepreddisone, paramethasone, prednisolone, prednisone, beclomethasone dipropionate, triamcinolone, as well as analogs, derivatives, salts, ions and complexes thereof.

In certain embodiments, the steroid is a hormone or a vitamin, as exemplified by pregnane, cholestane, ergostane, aldosterone, androsterone, calcidiol, calciol, calcitriol, calcipotriol, clomegestone, cholesterol, corticosterone, cortisol, cortisone, dihydrotestosterone, ergosterol, estradiol, estriol, estrone, ethinylestradiol, fusidic acid, lanosterol, prednisolone, prednisone, progesterone, spironolactone, timobesone and testosterone, as well as analogs, derivatives, salts, ions and complexes thereof.

In one embodiment, a subject is treated with a composition comprising a C1-C10 alcohol and an acid; and an anti-cancer therapy. Anti-cancer therapy includes, but is not limited to surgery, radiation, chemotherapeutics, anti-cancer drugs, and immunomodulators.

Illustrative examples of anti-cancer drugs include, but are not limited to: alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin and its pegylated compositions, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton, N.J.) and doxetaxel (TAXOTERE®., Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on cancers such as anti-estrogens including for example tamoxifen, raloxifene, aromatase inhibiting 4(5)-imidazoles, 4-hydroxytamoxifen, trioxifene, keoxifene. LY 117018, onapristone, and toremifene (Fareston); and anti-androgens such as flutamide, nilutamide, bicalutamide, leuprolide, and goserelin; DMSO, and pharmaceutically acceptable salts, acids or derivatives of any of the above.

Illustrative examples of immunomodulators include, but are not limited to: cyclosporine, tacrolimus, tresperimus, pimecrolimus, sirolimus, verolimus, laflunimus, laquinimod and imiquimod, as well as analogs, derivatives, salts, ions and complexes thereof.

In one embodiment, a subject is treated with a composition comprising a C1-C10 alcohol and an acid; and an anti-infective agent.

Illustrative examples of anti-infective agents include, but are not limited to: an antibiotic agent, an antibacterial agent, an antifungal agent, an agent that controls yeast, an antiviral agent and an antiparasitic agent. Exemplary anti-infective agents are exemplified by beta-lactam antibiotic, an aminoglycoside, an ansa-type antibiotic, an anthraquinone, an azole, metronidazole, an antibiotic glycopeptide, a macrolide, erythromycin, clindamycin, an antibiotic nucleoside, an antibiotic peptide, polymyxin B, an antibiotic polyene, an antibiotic polyether, an antibiotic quinolone, an antibiotic steroid, fucidic acid, mupirocin, chloramphenicol, a sulfonamide, tetracycline, an antibiotic metal, silver, copper, zinc, mercury, tin, lead, bismuth, cadmium, chromium, an oxidizing agent, iodine, iodate, a periodate, a hypochlorite, a permanganate, a substance that release free radicals and/or active oxygen, a cationic antimicrobial agent, a quaternary ammonium compound, a biguanide, chlorohexidine, a triguanide, a bisbiguanide, a polymeric biguanide and a naturally occurring antibiotic compound, as well as analogs, derivatives, salts, ions and complexes thereof.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### CERTAIN EMBODIMENTS

The present disclosure contemplates, among other things, the following numbered embodiments:
1. A method of reducing or decreasing a lesion on a surface of a subject comprising contacting the lesion and/or an area of the surface adjacent to the lesion with an effective amount of a composition comprising a C1-C10 alcohol and a sufficient amount of an acid to adjust the pH to below 5.5, wherein the lesion is not caused by a virus.
2. The method of embodiment 1, wherein the lesion comprises a gash, cut, puncture, abrasion, bump, blister, ulcer, allergic reaction, or burn on the surface.
3. The method of embodiment 1, wherein the lesion is the result of an insect bite or sting.
4. The method of embodiment 3, wherein the insect is a mosquito, flea, ant, wasp, bee, hornet, fly, louse, or bedbug.
5. The method of embodiment 1, wherein the lesion is the result of an arachnid bite or sting.
6. The method of embodiment 5, wherein the arachnid is a scorpion, spider, tick, or mite.
7. The method of embodiment 1, wherein the lesion is the result of an animal bite or sting.
8. The method of embodiment 7, wherein the animal is a mammal, a reptile, an amphibian, a cnidarian, or a mollusk.
9. The method of embodiment 8, wherein the mammal is a canine, feline, or rodent.
10. The method of embodiment 8, wherein the reptile is a snake or lizard.
11. The method of embodiment 8, wherein the amphibian is a frog, toad, or newt.
12. The method of embodiment 8, wherein the cnidarian is a jellyfish, coral, or sea anemone.
13. The method of embodiment 8, wherein the mollusk is a snail, octopus, or cone shell.
14. The method of embodiment 1, wherein the lesion is caused by a bacterium or fungus.
15. The method of embodiment 1, wherein the lesion is caused by contact with a plant.
16. The method of embodiment 15, wherein the plant is a Urticacea spp., a Euphorbiaceace spp., a Toxicodendron spp., a blue-green algae, poison ivy, poison oak, poison sumac, a stinging nettle, a dumb cane, a giant hogweed, a blisterbush, a cowhage, an Indian fig, an upas, a bull nettle, a ciega vista, a blinding tree, a manchineel tree, a sand-box tree, a stinging spurge, a noseburn, a giant stinging tree, a gympie, a nilgri nettle, a wood nettle, a fever nettle, a nettle tree, a cashew nut tree, a rengas, a holigarna, a Burma lac tree, a black poison wood, a marking nut tree, a ligas, or a Japanese lacquer tree.
17. The method of embodiment 1, wherein the lesion is caused by contact with a chemical irritant.
18. The method of embodiment 1, wherein the lesion is a benign lesion, a pre-cancerous lesion, or a cancerous lesion.
19. The method of embodiment 1, wherein the surface is selected from the group consisting of: a skin surface, a surface of a mucous membrane surface, and a surface of a nail.
20. The method of embodiment 1, wherein the surface is selected from the group consisting of: a tracheal surface, a nasal surface, a nasalpharyngeal surface, a pharyngeal surface, a vaginal surface, a gastrointestinal surface, a urothelial surface, and an artificially exposed mucosa or skin surface.
21. The method of embodiment 20, wherein the artificially exposed mucosa or skin surface is exposed by a procedure selected from the group consisting of: an incision, a colostomy, an iliostomy, an ileoconduit, a gastrostomy, a ureterostomy, a urethrostomy, a vesciostomy, a bile duct/common bile duct ostomy, and a tracheostomy.
22. The method of any one of embodiments 1-21, wherein the composition is formulated as an ointment, paste, drop, tincture, gel, cream, salve, lotion, lip balm, foam, spray, roll-on, or aerosol.
23. The method of any one of embodiments 1-22, wherein the C₁-C₁₀alcohol is methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, pop-2-in-1-ol, cyclohexane-1,2,3,4,5,6-hexol, or 2(2-propyl)-5-methyl-cyclohexane-1-ol.
24. The method of any one of embodiments 1-22, wherein the C1-C10 alcohol is a C1 to C3 monohydroxy alcohol or a C1 to C4 diol.
25. The method of embodiment 24, wherein the alcohol is selected from the group consisting of methanol, ethanol, 1-propanol, and 2-propanol.
26. The method of embodiment 24, wherein the alcohol is ethanol.
27. The method of embodiment 24, wherein the diol is selected from the group consisting of 2,3-butanediol, 1,2-butanediol, 1,3-butanediol, and 1,4-butanediol.
28. The method of any one of embodiments 1-27, wherein the acid is an organic acid.
29. The method of embodiment 28, wherein the organic acid comprises one or more of glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, or acetic acid.
30. The method of embodiment 28, wherein the organic acid comprises glycolic acid.
31. The method of any one of embodiments 1-27, wherein the acid is an inorganic acid.
32. The method of embodiment 31, wherein the inorganic acid comprises hydrochloric acid, boric acid, nitric acid, phosphoric acid, sulfuric acid, hydrobromic acid, hydroiodic acid, hydrosulfuric acid, or perchloric acid.
33. The method of any one of embodiments 1-32, wherein the composition is contacted to the lesion and/or to a surface adjacent to the lesion a plurality of times.
34. The method of any one of embodiments 1-33, wherein the composition is contacted to the lesion a plurality of times in an hour, a day, a week, or a month.
35. A method of reducing of decreasing inflammation of a surface of a subject comprising contacting the inflamed surface with an effective amount of a composition comprising a C₁-C₁₀ alcohol and a sufficient amount of an acid to adjust the pH to below 5.5, wherein the lesion is not caused by a virus.
36. The method of embodiment 35, wherein the inflammation is the result of a lesion.
37. The method of embodiments 36, wherein the lesion comprises a gash, cut, puncture, abrasion, bump, blister, ulcer, allergic reaction, or burn on the surface.
38. The method of embodiment 35, wherein the inflammation is the result of an insect bite or sting.
39. The method of embodiment 38, wherein the insect is a mosquito, flea, ant, wasp, bee, hornet, fly, louse, or bedbug.
40. The method of embodiment 35, wherein the inflammation is the result of an arachnid bite or sting.
41. The method of embodiment 40, wherein the arachnid is a scorpion, spider, tick, or mite.
42. The method of embodiment 35, wherein the inflammation is the result of an animal bite or sting.
43. The method of embodiment 42, wherein the animal is a mammal, a reptile, an amphibian, a cnidarian, or a mollusk.
44. The method of embodiment 43, wherein the mammal is a canine, feline, or rodent.
45. The method of embodiment 43, wherein the reptile is a snake or lizard.
46. The method of embodiment 43, wherein the amphibian is a frog, toad, or newt.
47. The method of embodiment 43, wherein the cnidarian is a jellyfish, coral, or sea anemone.
48. The method of embodiment 43, wherein the mollusk is a snail, octopus, or cone shell.
49. The method of embodiment 35, wherein the inflammation is caused by a bacterial or fungal infection.
50. The method of embodiment 35, wherein the inflammation is caused by contact with a plant.
51. The method of embodiment 50, wherein the plant is Urticacea spp., a Euphorbiaceace spp., a Toxicodendron spp., a blue-green algae, poison ivy, poison oak, poison sumac, a stinging nettle, a dumb cane, a giant hogweed, a blisterbush, a cowhage, an Indian fig, an upas, a bull nettle, a ciega vista, a blinding tree, a manchineel tree, a sand-box tree, a stinging spurge, a noseburn, a giant stinging tree, a gympie, a nilgri nettle, a wood nettle, a fever nettle, a nettle tree, a cashew nut tree, a rengas, a holigarna, a Burma lac tree, a black poison wood, a marking nut tree, a ligas, or a Japanese lacquer tree.
52. The method of embodiment 35, wherein the lesion is caused by contact with a chemical irritant.
53. The method of embodiment 35, wherein the inflammation is dermatitis, drug reaction, psoriasis or interstitial cystitis.
54. The method of embodiment 35, wherein the inflamed surface is selected from the group consisting of: a skin surface, a surface of a mucous membrane, and a surface of a nail.
55. The method of embodiment 35, wherein the inflamed surface is selected from the group consisting of: a tracheal surface, a nasal surface, a nasalpharyngeal surface, a pharyngeal surface, a vaginal surface, a gastro intestinal surface, a urothelial surface, and an artificially exposed mucosa or skin surface.
56. The method of embodiment 55, wherein the artificially exposed mucosa or skin surface is exposed by a procedure selected from the group consisting of: an incision, a colostomy, an iliostomy, an ileoconduit, a gastrostomy, a ureterostomy, a urethrostomy, a vesciostomy, a bile duct/common bile duct ostomy, and a tracheostomy.
57. The method of any one of embodiments 35-56, wherein the composition is formulated as an ointment, paste, drop, tincture, gel, cream, salve, lotion, lip balm, foam, spray, roll-on, or aerosol.
58. The method of any one of embodiments 35-57, wherein the C₁-C₁₀ alcohol is methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, or 2-(2-propyl)-5-methyl-cyclohexane-1-ol.
59. The method of any one of embodiments 35-57, wherein the C₁-C₁₀ alcohol is a C₁ to C₃ monohydroxy alcohol or a C₁ to C₄diol.
60. The method of embodiment 59, wherein the alcohol is selected from the group consisting of methanol, ethanol, 1-propanol, and 2-propanol.
61. The method of embodiment 60, wherein the alcohol is ethanol.
62. The method of embodiment 35-57, wherein the diol is selected from the group consisting of 2,3-butanediol, 1,2-butanediol, 1,3-butanediol, and 1,4-butanediol.
63. The method of any one of embodiments 35-62, wherein the acid is an organic acid.
64. The method of embodiment 63, wherein the organic acid comprises glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, or acetic acid.
65. The method of embodiment 64, wherein the organic acid is glycolic acid.
66. The method of any one of embodiments 35-62, wherein the acid is an inorganic acid.
67. The method of embodiment 66, wherein the inorganic acid comprises hydrochloric acid, boric acid, nitric acid, phosphoric acid, sulfuric acid, hydrobromic acid, hydroiodic acid, hydrosulfuric acid, or perchloric acid.
68. The method of any one of embodiments 35-67, wherein the composition is contacted to the surface a plurality of times.
69. The method of any one of embodiments 35-68, wherein the composition is contacted to the surface a plurality of times in an hour, a day, a week, or a month.
70. A method of reducing or decreasing irritation of a surface of a subject comprising contacting the irritated surface with an effective amount of a composition comprising a C₁-C₁₀ alcohol and a sufficient amount of an acid to adjust the pH to below 5.5, wherein the lesion is not caused by a virus.
71. The method of embodiment 70, wherein the irritation is the result of a lesion.
72. The method of embodiments 71, wherein the lesion comprises a gash, cut, puncture, abrasion, bump, blister, ulcer, allergic reaction, or burn on the surface.
73. The method of embodiment 70, wherein the irritation is the result of an insect bite or sting.
74. The method of embodiment 73, wherein the insect is a mosquito, flea, ant, wasp, bee, hornet, fly, louse, or bedbug.
75. The method of embodiment 70, wherein the irritation is the result of an arachnid bite or sting.
76. The method of embodiment 75, wherein the arachnid is a scorpion, spider, tick, or mite.
77. The method of embodiment 70, wherein the irritation the result of an animal bite or sting.
78. The method of embodiment 77, wherein the animal is a mammal, a reptile, an amphibian, a cnidarian, or a mollusk.
79. The method of embodiment 77, wherein the reptile is a snake or lizard.
80. The method of embodiment 77, wherein the amphibian is a frog, toad, or newt.
81. The method of embodiment 77, wherein the cnidarian is a jellyfish, coral, or sea anemone.
82. The method of embodiment 77, wherein the mollusk is a snail, octopus, or cone shell.
83. The method of embodiment 70, wherein the irritation is caused by a bacterial or fungal infection.
84. The method of embodiment 70, wherein the irritation is caused by a chemical irritant.
85. The method of embodiment 70, wherein the irritation is caused by contact with a plant.
86. The method of embodiment 77, wherein the plant is Urticacea spp., a Euphorbiaceace spp., a Toxicodendron spp., a blue-green algae, poison ivy, poison oak, poison sumac, a stinging nettle, a dumb cane, a giant hogweed, a blisterbush, a cowhage, an Indian fig, an upas, a bull nettle, a ciega vista, a blinding tree, a manchineel tree, a sand-box tree, a stinging spurge, a noseburn, a giant stinging tree, a gympie, a nilgri nettle, a wood nettle, a fever nettle, a nettle tree, a cashew nut tree, a rengas, a holigarna, a Burma lac tree, a black poison wooxd, a marking nut tree, a ligas, or a Japanese lacquer tree.
87. The method of embodiment 70, wherein the irritation is caused by inflammation.
88. The method of embodiment 87, wherein the inflammation is dermatitis, drug reaction, psoriasis or interstitial cystitis.
89. The method of embodiment 70, wherein the irritated surface is selected from the group consisting of: a skin surface, a surface of a mucous membrane, and a surface of a nail.
90. The method of embodiment 70, wherein the irritated surface is selected from the group consisting of: a tracheal surface, a nasal surface, a nasalpharyngeal surface, a pharyngeal surface, a vaginal surface, a gastrointestinal surface, a urothelial surface, and an artificially exposed mucosa or skin surface.
91. The method of embodiment 90, wherein the artificially exposed mucosa or skin surface is exposed by a procedure selected from the group consisting of: an incision, a colostomy, an iliostomy, an ileoconduit, a gastrostomy, a ureterostomy, a urethrostomy, a vesciostomy, a bile duct/common bile duct ostomy, and a tracheostomy.
92. The method of any one of embodiments 70-91, wherein the composition is formulated as an ointment, paste, drop, tincture, gel, cream, salve, lotion, lip balm, foam, spray, roll-on, or aerosol.
93. The method of any one of embodiments 70-92, wherein the C₁-C₁₀ alcohol is methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, or 2-(2-propyl)-5-methyl-cyclohexane-1-ol.
94. The method of any one of embodiments 70-93, wherein the C₁-C₁₀alcohol is a C₁ to C₃ monohydroxy alcohol or a C₁ to C₄ diol.
95. The method of embodiment 94, wherein the alcohol is selected from the group consisting of methanol, ethanol, 1-propanol, and 2-propanol.
96. The method of embodiment 95, wherein the alcohol is ethanol.
97. The method of embodiment 94, wherein the diol is selected from the group consisting of 2,3-butanediol, 1,2-butanediol, 1,3-butaneciol, and 1,4-butanediol.
98. The method of any one of embodiments 70-97, wherein the acid is an organic acid.
99. The method of embodiment 98, wherein the organic acid comprises one or more of glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, or acetic acid.
100. The method of embodiment 99, wherein the organic acid is glycolic acid.
101. The method of any one of embodiments 70-97, wherein the acid is an inorganic acid.
102. The method of embodiment 101, wherein the inorganic acid comprises hydrochloric acid, boric acid, nitric acid, phosphoric acid, sulfuric acid, hydrobromic acid, hydroiodic acid, hydrosulfuric acid, or perchloric acid.
103. The method of any one of embodiments 70-102, wherein the composition is contacted to the surface a plurality of times.
104. The method of any one of embodiments 70-103, wherein the composition is contacted to the surface a plurality of times in an hour, a day, a week, or a month.
105. A method of reducing or decreasing a bacterial or fungal infection in a subject comprising contacting an infected surface with an effective amount of a composition comprising a C₁-C₁₀alcohol and a sufficient amount of an acid to adjust the pH to below 5.5, wherein the lesion is not caused by a virus.
106. The method of embodiment 105, wherein the infection is a bacterial infection.
107. The method of embodiment 106, wherein the bacterial infection is caused by a gram negative bacterium.
108. The method of embodiment 106, wherein the bacterial infection is caused by a gram positive bacterium.
109. The method of embodiment 106, wherein the bacterial infection is caused by a Mycobacterium spp., a Pneumococcus spp., an Escherichia spp., a Campylobacter spp., a Corynebacterium spp., a Clostridium spp., a Streptococcus spp., a Staphylococcus spp., a Pseudomonas spp., a Shigella spp., a Treponema spp., or a Salmonella spp.
110. The method of embodiment 105, wherein the infection is a fungal infection.
111. The method of embodiment 110, wherein the fungal infection is caused by:
   (a) a species of yeast; or
   (b) an Aspergillis spp., a Blastomyces spp., a Candida spp., a Coccicioides spp., a Cryptococcus spp., dermatophytes, a Tinea spp., a Trichophyton spp., a Microsporum spp., a Fusarium spp., a Histoplasma spp., a Mucoromycotina spp., a Pneumocystis spp., a Sporothrix spp., an Exserophilum spp., or a Cladosporium spp.
112. The method of embodiment 105, wherein the infected surface is selected from the group consisting of a skin surface, a surface of a mucous membrane, and a surface of a nail.
113. The method of embodiment 105, wherein the infected surface is selected from the group consisting of: a tracheal surface, a nasal surface, a nasalpharyngeal surface, a pharyngeal surface, a vaginal surface, a gastrointestinal surface, a urothelial surface, and an artificially exposed mucosa or skin surface.
114. The method of embodiment 113, wherein the artificially exposed mucosa or skin surface is exposed by a procedure selected from the group consisting of: an incision, a colostomy, an iliostomy,an ileoconduit, a gastrostomy, a ureterostomy, a urethrostomy, a vesciostomy, a bile duct/common bile duct ostomy, and a tracheostomy.
115. The method of any one of embodiments 105-114, wherein the composition is formulated as an ointment, paste, drop, tincture, gel, cream, salve, lotion, lip balm, foam, spray, roll-on, or aerosol.
116. The method of any one of embodiments 105-115, wherein the C₁-C₁₀alcohol is methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, or 2-(2-propyl)-5-methyl-cyclohexane-1-ol.
117. The method of any one of embodiments 105-115, wherein the C₁-C₁₀alcohol is a C₁ to C₃ monohydroxy alcohol or a C₁ to C₄ diol.
118. The method of embodiment 117, wherein the alcohol is selected from the group consisting of methanol, ethanol, 1-propanol, and 2-propanol.
119. The method of embodiment 118, wherein the alcohol is ethanol.
120. The method of embodiment 117, wherein the diol is selected from the group consisting of 2,3-butanediol, 1,2-butanediol, 1,3-butanediol, and 1,4-butanediol.
121. The method of any one of embodiments 105-120, wherein the acid is an organic acid.
122. The method of embodiment 121, wherein the organic acid comprises one or more of glycolic acid, lactic acid, squaric acid, succinic acid, malic acid, citric acid, formic acid, hydroxycinnamic acid, ferulic acid, oxalic acid, uric acid, 4-dihydroxy benzoic acid, p-hydroxy benzoic acid, vanillic acid, p-coumaric acid, gallic acid, syringic acid, salicylic acid, luteic acid, eudesmic acid, or acetic acid.
123. The method of any one of embodiments 105-120, wherein the acid is an inorganic acid.
124. The method of embodiment 123, wherein the inorganic acid comprises hydrochloric acid, boric acid, nitric acid, phosphoric acid, sulfuric acid, hydrobromic acid, hydroiodic acid, hydrosulfuric acid, or perchloric acid.
125. The method of any one of embodiments 105-124, wherein the composition is contacted to the surface a plurality of times.
126. The method of any one of embodiments 105-125, wherein the composition is contacted to the surface a plurality of times in an hour, a day, a week, or a month.
127. A method of reducing or decreasing pain in a subject wherein the pain is caused by a lesion, inflammation, irritation, or bacterial or fungal infection of a surface of a subject comprising contacting the painful surface with an effective amount of a composition comprising a C₁-C₁₀ alcohol and a sufficient amount of an acid to adjust the pH to below 5.5, wherein the lesion is not caused by a virus.
128. The method of embodiment 127, wherein the pain is the result of a lesion of the surface.
129. The method of embodiments 128, wherein the lesion comprises a gash, cut, puncture, abrasion, bump, blister, ulcer, allergic reaction, or burn on the surface.
130. The method of embodiment 127, wherein the pain is the result of inflammation of the surface.
131. The method of embodiment 127, wherein the pain is the result of irritation of the surface.
132. The method of embodiment 127, wherein the pain is the result of an insect bite or sting.
133. The method of embodiment 132, wherein the insect is a mosquito, flea, ant, wasp, bee, hornet, fly, louse, or bedbug.
134. The method of embodiment 127, wherein the pain is the result of an arachnid bite or sting.
135. The method of embodiment 134, wherein the arachnid is a scorpion, spider, tick, or mite.
136. The method of embodiment 127, wherein the pain is the result of an animal bite or sting.
137. The method of embodiment 136, wherein the animal is a mammal, a reptile, an amphibian, a cnidarian, or a mollusk.
138. The method of embodiment 137, wherein the reptile is a snake or lizard.
139. The method of embodiment 137, wherein the amphibian is a frog, toad, or newt.
140. The method of embodiment 137, wherein the cnidarian is a jellyfish, coral, or sea anemone.
141. The method of embodiment 137, wherein the mollusk is a snail, octopus, or cone shell.
142. The method of embodiment 127, wherein the pain is caused by a bacterial or fungal infection.
143. The method of embodiment 127, wherein the pain is caused by a chemical irritant.
144. The method of embodiment 127, wherein the pain is caused by contact with a plant.
145. The method of embodiment 144, wherein the plant is Urticacea spp., a Euphorbiaceace spp., a Toxicodendron spp., a blue-green algae, poison ivy, poison oak, poison sumac, a stinging nettle, a dumb cane, a giant hogweed, a blisterbush, a cowhage, an Indian fig, an upas, a bull nettle, a ciega vista, a blinding tree, a manchineel tree, a sand-box tree, a stinging spurge, a nosebum, a giant stinging tree, a gympie, a nilgri nettle, a wood nettle, a fever nettle, a nettle tree, a cashew nut tree, a rengas, a holigarna, a Burma lac tree, a black poison wood, a marking nut tree, a ligas, or a Japanese lacquer tree.
146. The method of embodiment 127, wherein the infection is a bacterial infection.
147. The method of embodiment 146, wherein the bacterial infection is caused by a gram negative bacterium.
148. The method of embodiment 146, wherein the bacterial infection is caused by a gram positive bacterium.
149. The method of embodiment 148, wherein the bacterial infection is caused by a Mycobacterium spp., a Pneumococcus spp., an Escherichia spp., a Campylobacter spp., a Corynebacterium spp., a Clostridium spp., a Streptococcus spp., a Staphylococcus spp., a Pseudomonas spp., a Shigella spp., a Treponema spp., or a Salmonella spp.
150. The method of embodiment 127, wherein the infection is a fungal infection.
151. The method of embodiment 150, wherein the fungal infection is caused by:
   (a) a species of yeast; or
   (b) an Aspergillis spp., a Blastomyces spp., a Candida spp., a Coccicioides spp., a Cryptococcus spp., dermatophytes, a Tinea spp., a Trichophyton spp., a Microsporum spp., a Fusarium spp., a Histoplasma spp., a Mucoromycotina spp., a Pneumocystis spp., a Sporothrix spp., an Exserophilum spp., or a Cladosporium spp.
152. The method of embodiment 127, wherein the painful surface is selected from the group consisting of: a skin surface, a surface of a mucous membrane, and a surface of a nail.
153. The method of embodiment 127, wherein the surface is selected from the group consisting of: a tracheal surface, a nasal surface, a nasalpharyngeal surface, a pharyngeal surface, a vaginal surface, a gastrointestinal surface, a urothelial surface, and an artificially exposed mucosa or skin surface.
154. The method of embodiment 153, wherein the artificially exposed mucosa or skin surface is exposed by a procedure selected from the group consisting of: an incision, a colostomy, an iliostomy, an ileoconduit, a gastrostomy, a ureterostomy, a urethrostomy, a vesciostomy, a bile duct/common bile duct ostomy, and a tracheostomy.
155. The method of any one of embodiments 127-154, wherein the composition is formulated as an ointment, paste, drop, tincture, gel, cream, salve, lotion, lip balm, foam, spray, roll-on, or aerosol.
156. The method of any one of embodiments 127-155, wherein the C₁-C₁₀ alcohol is methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, or 2-(2-propyl)-5-methyl-cyclohexane-1-ol.
157. The method of any one of embodiments 127-155, wherein the C₁-C₁₀ alcohol is a C₁ to C₃ monohydroxy alcohol or a C₁ to C₄ diol.
158. The method of embodiment 157, wherein the alcohol is selected from the group consisting of methanol, ethanol, 1-propanol, and 2-propanol.
159. The method according to embodiment 158, wherein the alcohol is ethanol.
160. The method of embodiment 157, wherein the diol is selected from the group consisting of 2,3-butanediol, 1,2-butanediol, 1,3-butanediol, and 1,4-butanediol.
161. The method of any one of embodiments 127-160, wherein the acid is an organic acid.
162. The method of embodiment 161, wherein the organic acid comprises one or more of glycolic acid, lactic acid, squaric acid, squaric acid, succinic acid, malic acid, citric acid, formic acid, hydroxycinnamic acid, ferulic acid, oxalic acid, uric acid, 4-dihydroxy benzoic acid, p-hydroxybenzoic acid, vanillic acid, p-coumaric acid, gallic acid, syringic acid, salicylic acid, luteic acid, eudesmic acid, or acetic acid.
163. The method of embodiment 162, wherein the organic acid is glycolic acid.
164. The method of any one of embodiments 127-160, wherein the acid is an inorganic acid.
165. The method of embodiment 164, wherein the inorganic acid comprises hydrochloric acid, boric acid, nitric acid, phosphoric acid, sulfuric acid, hydrobromic acid, hydroiodic acid, hydrosulfuric acid, or perchloric acid.
166. The method of any one of embodiments 127-165, wherein the composition is contacted to the surface a plurality of times.
167. The method of any one of embodiments 127-166, wherein the composition is contacted to the surface a plurality of times in an hour, a (lay, a week, or a month.
168. The method of any one of embodiments 1-167, wherein the composition consists essentially of an alcohol and an acid.
169. The method of any one of embodiments 1-167, wherein the composition consists of an alcohol and an acid as the active components.
170. The method of any one of embodiments 1-169, wherein the pH of the composition ranges from pH 2.0 to about pH 5.5.
171. The method of embodiment 170, wherein the pH of the composition ranges is about pH 3.5 or lower.
172. The method of embodiment 170, wherein the pH of the composition is about pH 3.0 or lower.
173. The method of embodiment 170, wherein the pH of the composition is about pH 2.5 or lower.
174. The method according to any one of embodiments 1-173, wherein the alcohol concentration in the composition ranges from about 0.2% by volume up to about 40% by volume.
175. The method of embodiment 174, wherein the alcohol concentration in the composition ranges from about 0.5% by volume up to about 20% by volume.
176. The method of embodiment 174, wherein the alcohol concentration in the composition ranges from about 1% by volume up to about 15% by volume.
177. The method according to any one of embodiments 1-169, wherein the composition comprises:
   about 0.2% to about 15% ethanol or from about 1% or about 2% to about 12% ethanol; and
   about 0.1% w/v to about 5% w/v glycolic acid, or about 0.2% w/v to about 4% w/v glycolic acid, or about 0.2% w/v to about 4% w/v glycolic acid, or about 0.3% w/v to about 3% w/v glycolic acid, or about 0.4% w/v to about 2% w/v glycolic acid, or about 0.5% w/v to about 1% w/v glycolic acid.
178. The method of embodiment 177, wherein the composition comprises about 10% ethanol; and about 0.6% w/v glycolic acid.
179. The method according to any one of embodiments 1-178, wherein the composition comprises a pharmaceutically acceptable excipient.
180. The method according to any one of embodiments 1-179, wherein the composition excludes an amphoteric or pseudoamphoteric compound.
181. The method according to any one of embodiments 1-179, wherein the composition excludes an amino acid and/or a peptide.
182. The method according to any one of embliments 1-179, wherein the composition excludes a dipeptide and/or a tripeptide.
183. The method according to any one of embodiments 1-179, wherein the composition excludes an imidazoline amphoteric and/or a lecithin amphoteric.
184. The method according to any one of embodiments 1-179, wherein the composition excludes one or more compounds selected from the group consisting of cocoamphoglycine, cocoamphoproprionate, cocoamphopropylsulfonate, phosphatidyl ethanolamine, phosphatidyl serine, and sphingomyelin.
185. The method according to any one of embodiments 1-184, wherein the composition is formulated as a unit dosage formulation.
186. A method for treating an inflammatory or autoimrmune lesion, comprising contacting the lesion with an effective amount of an acidic composition comprising a C1-C10 alcohol.
187. The method of embodiment 186, wherein the acidic composition comprises an amount of an acid sufficient to provide a pH of less than 6.5.
188. The method of embodiment 186, wherein the acidic composition comprises an amount of an acid sufficient to provide a pH of less than less than 6.
189. The method of embodiment 186, wherein the acidic composition comprises an amount of an acid sufficient to provide a pH of 5.5.
190. The method of embodiment 186, wherein the acidic composition comprises an amount of an acid sufficient to provide a pH of from about 4 to about 6.
191. The method of embodiment 186, wherein the acidic composition comprises an amount of an acid sufficient to provide a pH of from about 5 to about 6.
192. The method of embodiment 186, wherein the acidic composition comprises an amount of an acid sufficient to provide a pH of less than 5.5.
193. The method of any one of embodiments 186-192, wherein the inflammatory or autoimmune lesion is associate with a disorder selected from psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne and alopecia areata.
194. The method of any one of embodiments 1-32, wherein the surface is an oral surface.
195. The method of embodiment 194, wherein the oral surface is a gum surface, tooth surface, or both.
196. The method of embodiment 194 or 195, wherein the oral surface is in a subject having periodontal disease, dental caries, or both.
197. The method of any one of embodiments 194-197, wherein the oral surface is infected with Streptococcus mutans.

### EXAMPLES

### Example 1 Glycolic Acid:Ethanol Composition Promotes Wound Healing

### BACKGROUND

Groups of wild type 7-8 week old mice (n=6/group) were anesthetized using Ketamine/xylazine i.p. at 100/10 mg/kg body weight and shaved with electric clippers. Following the induction of anesthesia and the loss of consciousness, bland ophthalmic ointment was applied to the eyes of the mouse to prevent drying of the cornea and all animals were carefully monitored for anesthetic depth. The anesthesia time per mouse was 5-10 minutes, 6 mice were in the treatment arm, and 6 mice in the untreated/placebo arm, for a total of 12 mice, 2 full-thickness excisional wounds (4 mm in size) were generated in the back skin of anesthetized control and treated female mice using sterile biopsy punches as has been previously described (Liang et al., 2012; Ganguli-Indra et al., 2014).

### Treatment

The wounds of each mouse were sprayed to saturation either with Placebo or glycolic acid:ethanol composition (pH<4.5) for 4 days. The process was repeated every 20 minutes, for a total of 3 treatments. Then, this 3× treatment protocol was repeated every 6 hours, i.e., 4 times a day, for 4 days. Mice were handled by the same personnel during treatment and data recording for consistency in handling and treatment. The mice were monitored daily, 7 days a week, until wound closure, for signs of pain and discomfort, and no secondary infections were observed in treatment groups maintained in micro-isolators. All mice in the treatment and the placebo group were euthanized and sample harvested at the end of 13 days of data recording. Wound tissue, including 5 mm surrounding wound margin skin, were snap frozen in liquid nitrogen and stored at −80° C.

### Results

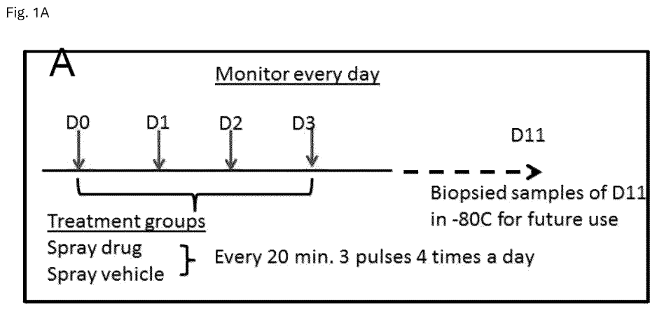

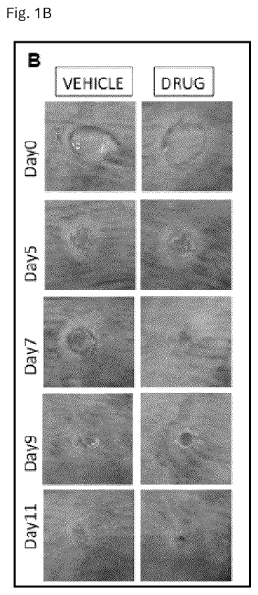

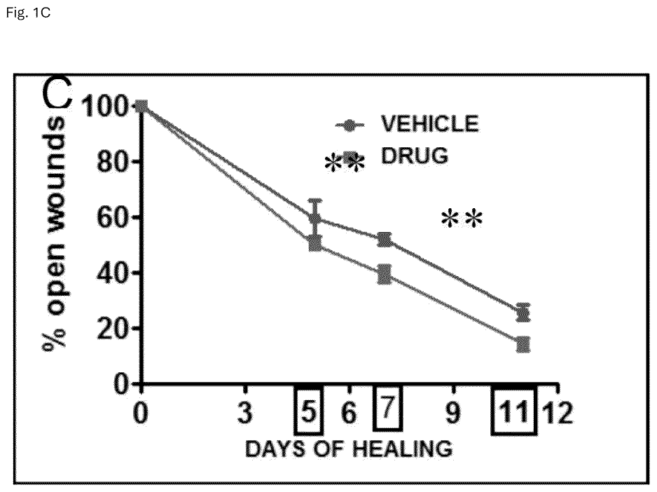

The healing process at days 1, 3, 5, 7, 9, 11 and 13 post-injury was recorded using a digital camera and analyzed for wound closure rate (FIG. 1A). Surface area of the wounds were quantitated by using Zeiss scientific image analysis software (Carl Zeiss Inc.) and plotted as a percentage of open wounds (FIG. 1B). In order to obtain the % of open wound, current surface area of each wound was divided by the initial surface area at any given timepoints, then this data was combined to give one data point for the treated and one data point for the untreated and these data points were graphed with their respective standard deviation.

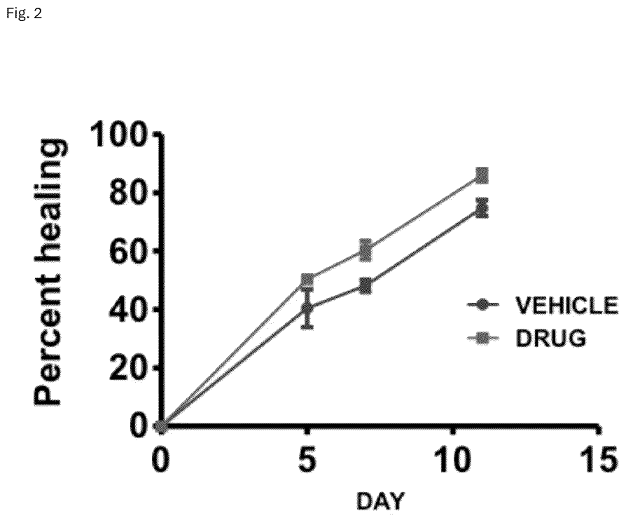

At days 5, 7 and 11 post wounding and following treatment with drug/vehicle, the rate of wound healing in the drug treated arm was 10%, 12% and 12% faster than the vehicle treated arm, respectively (FIG. 2). There was no observed signs of pain and discomfort, and no secondary infections were observed in treatment groups of mice at all timepoints.

### REFERENCES

1. Liang X, Bhattacharya S, Bajaj G, Guha G, Wang Z, Jang H S, Leid M, Indra A K, Ganguli-Indra G. (2012). Delayed Cutaneous Wound Healing and Aberrant Expression of Hair Follicle Stem Cell Markers in Mice Selectively Lacking Ctip2 in Epidermis. PLoS One, 2012; 7(2):e29999. Epub 2012 Feb. 21. PMCID3283611.
2. Gitali Ganguli-Indra (2014). Protocol for cutaneous wound healing assay in a murine model. Stem Cells and Tissue Repair. Methods in Molecular Biology Volume 1210, 2014, pp 151-159.

### Example 2 Glycolic Acid:Ethanol Composition Promotes Wound Healing

### Background

Groups of wild type 6-7 week old mice (n=8/group) were anesthetized using Ketamine/xylazine i.p. at 100/10 mg/kg body weight and shaved with electric clippers. Following the induction of anesthesia and the loss of consciousness, bland ophthalmic ointment was applied to the eyes of the mouse to prevent drying of the cornea and all animals were carefully monitored for anesthetic depth. The anesthesia time per mouse was 5-10 minutes, 8 mice were in the treatment arm, and 6 mice in the untreated/placebo arm, for a total of 16 mice.

2 full-thickness excisional wounds (4 mm in size) were generated in the back skin of anesthetized control and treated female mice using sterile biopsy punches as has been previously described (Liang et al., 2012; Ganguli-Indra et al., 2014).

### Treatment

The wounds of each mouse were sprayed to saturation either with Placebo or gylcolic acid:ethanol composition (pH<4.5) for 4 days. The process was repeated every 20 minutes, for a total of 3 treatments. Then, this 3× treatment protocol was repeated every 6 hours, i.e., 4 times a day, for 4 days. Mice were handled by the same personnel during treatment and data recording for consistency in handling and treatment. The mice were monitored daily, 7 days a week, until wound closure, for signs of pain and discomfort, and no secondary infections were observed in treatment groups maintained in micro-isolators. All mice in the treatment and the placebo group were euthanized and sample harvested at the end of 13 days of data recording. Wound tissue, including 5 mm surrounding wound margin skin, were snap frozen in liquid nitrogen and stored at −80° C.

### Results

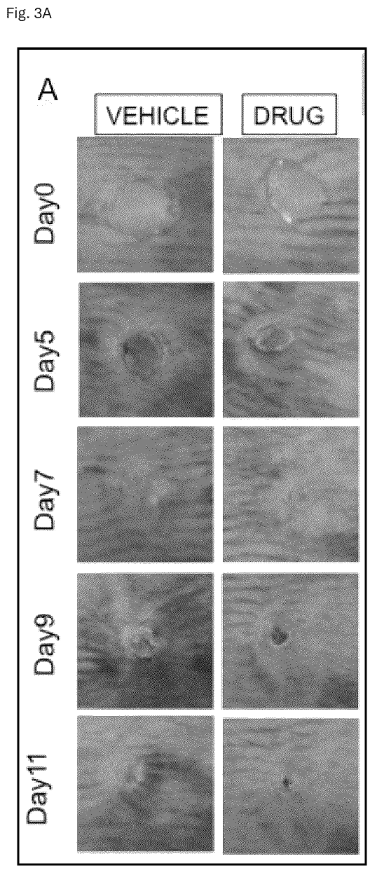

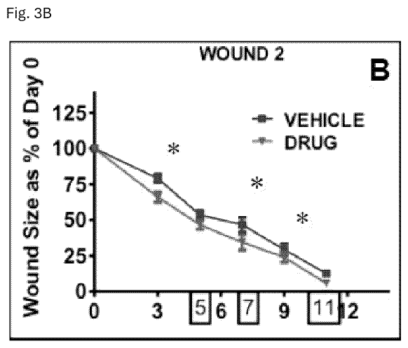

The healing process at days 1, 3, 5, 7, 9 and 11 post-injury were recorded using a digital camera and analyzed for wound closure rate for each time point and treatment (FIG. 3A) as described above. Surface area of the wounds were quantitated by using Zeiss scientific image analysis software (Carl Zeiss Inc.) and plotted as a percentage of open wounds (FIG. 3B). In order to obtain the % of open wound, current surface area of each wound was divided by the initial surface area at a given timepoint, then this data was combined to give one data point for the treated and one data point for the untreated and these data points were graphed with their respective standard deviation.

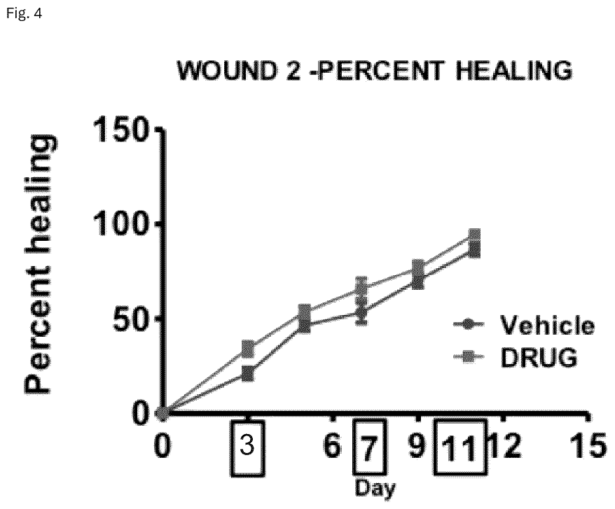

At days 3, 7 and 11 post wounding and following treatment with drug/vehicle, the rate of wound healing in the drug treated arm was 13%, 13% and 3% faster than the vehicle treated arm, respectively (see FIG. 4). There was no observed signs of pain and discomfort, and no secondary infections were observed in treatment groups of mice at all timepoints.

### Conclusions from Examples 1 and 2

The results from these two independent studies and gross morphological analysis show that topical treatment with the present acidic composition accelerated wound closure and significantly enhanced healing of full-thickness skin wounds in vivo in adult mice. The graphs of the treated and untreated groups diverge prior to day 5 (in exp 1) and day 3 (in exp 2) and then run parallel so as to indicate that the drug effects relate to the inflammation process, in addition to the proliferation and remodeling steps of the healing.

### Example 3 Efficacy of Glycolic Acid:Ethanol Composition in Reducing Inflammation in a Xenograft Model

### Treatment—Part I

Human adult skin grafts (1-2 cm² in size) were engrafted onto the dorsal skin of immunocompromised SCID mice When grafts had taken and scabs had fallen, freshly-isolated PBMCs from healthy donors were injected intraperitoneally. Blood was collected every week for 3 weeks from the tail vein to assess the presence of PBMCs, and mice were euthanized at two time points (day 14 and day 21) post-PBMC injection. The primary endpoint is the presence of human immunoregulatory cell types; CD4, CD3, IL17 and FOXP3, in skin by immunohistochemical (IHC) labelling. The secondary endpoint is the presence of human and mouse CD45, and human CD3, in peripheral blood as analyzed by FACS.

### Treatment—Part II

The efficacy of using glycolic acid:ethanol compositions (pH<5.5, such as 2.5) in the xenograft model to reduce inflammation was measured. The primary endpoint is the presence of human immunoregulatory cell types; CD4, CDS, ILl7 and FOXP3, in skin by immunohistochemical (IHC) labelling. The secondary endpoint is the presence of human and mouse CD45, and human CD3, in peripheral blood as analyzed by FACS.

### Methods—Part I

Twenty female SCID mice were purchased at 4-6 weeks of age. All mice were held in individually ventilated cages (IVCs) in an SPF barrier unit and all procedures were certified according to the UK Home Office Animals (Scientific Procedures) Act 1986. Animals were acclimatized for 1-2 weeks, prior to use.

Normal breast or abdominal skin was obtained from a single donor. All mice were anaesthetized and administered with buprenorphine analgesia and 1-2 cm² pieces of human skin were xenografted onto the dorsum of each of the SCID mice. Depending on size of skin, up to 20 mice received xenografts. A similar piece of donor control human skin (not xenografted) and one piece of excised mouse skin (from a donor acceptance site) was taken, bisected, half snap frozen and half formalin fixed. Wounds were allowed to heal for 3 weeks and then ten of the twenty animals were then injected intraperitoneally (i.p.) with 150×10⁶ PBMCs each sourced from human blood donors (PBMCs not sourced from the skin donor). Mice are then divided into the following groups:
Group 1: Skin graft plus PBMCs; euthanize day 14 post-PBMC administration
Group 2: Skin graft minus PBMCs; euthanize day 14 post-PBMC administration
Group 3: Skin graft plus PBMCs; euthanize day 21 post-PBMC administration
Group 4: Skin graft minus PBMCs; euthanize day 21 post-PBMC administration
   Following skin engraftment, mice were weighed three times a week. Following PBMC injection, mice were weighed daily. Any irritation of the skin was noted.

If an animal becomes unwell, any treatment of that animal is suspended. If there is no recovery the animal is euthanized. Any animal demonstrating more than 15% weight loss is considered unwell. Any animal is euthanized if the weight loss is greater than 20%. Animal well-being is inspected daily.

Blood is collected from all mice, from a tail vein bleed, on days 7, 14 and 21 post-PBMC injection and assessed for human cell populations by FACS for the following cell markers:
1. Human and mouse CD45
2. Human CD3
   Fourteen and twenty one days following injection of the PBMCs, mice were euthanized, as outlined above.

At the time of euthanasia, the skin graft was excised, along with some adjacent mouse skin (as one complete piece) and bisected. One piece of the skin graft was fixed for 24 hours at room temperature in phosphate buffered formalin (pH 7.0-7.4), transferred to 70% ethanol then processed and paraffin embedded using standard methods, to create a total of 20 formalin-fixed paraffin-embedded (FFPE) blocks. The other piece of the bisected skin graft was snap frozen. In addition, blood was collected by cardiac puncture and serum prepared, frozen and stored.

Antibodies (one per marker) was used to detect the following human markers:
1. CD4
2. CDS
3. IL17
4. FOXP3
   The antibody labeling protocol for use on FFPE inflamed human tissue (human psoriatic skin or human IBD tissue) was optimized for each antibody. Negative control slides were included to confirm specificity (e.g., non-inflamed human tissue sections). A range of antibody concentrations, antigen retrieval, blocking and washing techniques were tested, as necessary. DAB chromagen was used to visualize binding.

After the antibody labeling protocols were optimized, eight non-serial sections were cut from each FFPE skin graft, mounted onto 1 slide per 2 sections (4 slides in total per graft) and were immunohistochemically labeled using the optimized protocols for CD4, CDS, IL17 and FOXP3 (1 slide per graft for each antibody). Negative controls included a tissue section minus the primary antibody, a section of mouse skin and a section of normal (non-inflamed human tissue). A positive control included inflamed human tissue sections, as used in the optimization stage.

### Methods—Part II

Twenty female SCID mice were purchased at 4-6 weeks of age. All mice were held in individually ventilated cages (IVCs) in an SPF barrier unit and all procedures were certified according to the UK Home Office Animals (Scientific Procedures) Act 1986. Animals were acclimatized for 1-2 weeks, prior to use.

Normal breast or abdominal skin was obtained from a single donor. All mice were anesthetized and administered with buprenorphine analgesia and 1-2 cm² pieces of human skin were xenografted onto the dorsum of each of the SCID mice. A similar piece of donor control human skin (not xenografted) and one piece of excised mouse skin (from a donor acceptance site) were taken, bisected, half snap frozen and half formalin fixed.

Wounds were allowed to heal for 3 weeks and then ten of the twenty animals were then injected intraperitoneally (i.p.) with 150×10′ PBMCs each sourced from human blood donors (PBMCs were not be sourced from the skin donor).

Following injection of the PBMCs, glycolic acid:ethanol compositions (pH 2.5) was administered. The mice were divided into the following groups and are administered the glycolic acid:ethanol compositions (pH 2.5) or vehicle as follows:
Vehicle—12 times daily, for 7 days, topical administration using a spray—i.e., every 6 hours, vehicle is administered 3 times in an hour (every 20 mins; e.g., at 1:00 am, 1:20 am, 1:40 am; 7:00 am, 7:20 am, 7:40 am; 1:00 pm, 1:20 pm, 1:40 pm; and 7:00 pm, 7:20 pm, 7:40 pm).
glycolic acid:ethanol compositions (pH 2.5)—12 times daily, for 7 days, topical administration using a spray—i.e. every 6 hours, test item is administered 3 times in an hour (every 20 mins; e.g. at 1:00 am, 1:20 am, 1:40 am; 7:00 am, 7:20 am, 7:40 am; 1:00 pm, 1:20 pm, 1:40 pm; and 7:00 pm, 7:20 pm, 7:40 pm).

The effect of the glycolic acid:ethanol compositions (pH 2.5) on reducing inflammation was assessed.

Following skin engraftment, mice were weighed three times a week. Following PBMC injection and during administration of the compounds, mice are weighed daily. Any irritation of the skin is noted. If an animal becomes unwell, any treatment of that animal is suspended. If there is no recovery the animal is euthanized. Any animal demonstrating more than 15% weight loss is considered unwell. Any animal is euthanized if the weight loss is greater than 20%. Animal well-being is inspected daily.

All animals were euthanized, on day 7 following commencement of test item administration (day 21 post-PBMC injection) with a single terminal bleed and spleen was collected for Fluorescent Activated Cell sorting (FACS) analysis. At the time of euthanasia, the skin graft was excised, along with some adjacent mouse skin (as one complete piece) and bisected. One piece of the skin graft was fixed for 24 hours at room temperature in phosphate buffered formalin (pH 7.0-7.4), transferred to 70% ethanol then processed and paraffin embedded, to create a total of 20 FFPE blocks. The other piece of the bisected skin graft was snap frozen. In addition, blood was collected and serum prepared, frozen and stored. Eight non-serial sections were cut from each FFPE skin graft, mounted onto 1 slide 10 per 2 sections (4 slides in total per graft) and are labeled for human CD4, CD8, IL17 and FOXP3 (1 slide per graft for each antibody). Negative controls included a tissue section minus the primary antibody, a section of mouse skin and a section of normal (non-inflamed human tissue). A positive control included inflamed human tissue sections. Collected blood assessed for cell populations by FACS for the following cell markers: 1. Human and mouse CD45 2. Human CD3 Skin grafts were assessed for T-cell populations (CD4 and CD8) and inflammation markers (FOXP3) using immunohistochemistry (IHC) protocols standardized during the study to label human CD4, CD8 and FOXP3.

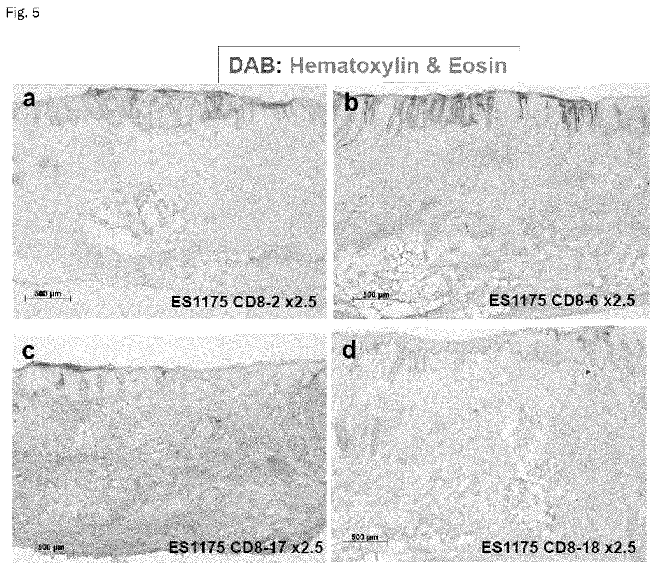

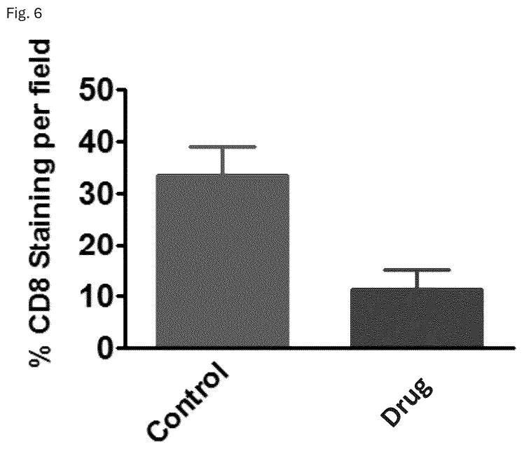

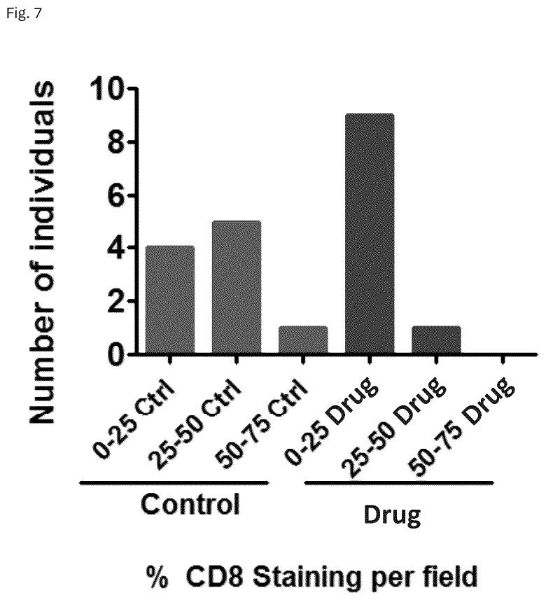

By IHC analyses, all the markers were found to be expressed in both vehicle—as well as treated animals, although there was variability in the staining pattern and intensity between the two groups. Analysis of spleenocyte populations by FACS sorting at the end of the study revealed a reduction in peripheral populations of CD3+ human T-cells in treated (with the present acidic composition), versus vehicle-administered animals. By IHC analyses, successful infiltration of CD4+ Thelper cells and CD8+ Cytotoxic T-cells, as well as anti-inflammatory transcription factor FOXP3 was observed in the human skin xenografts in SCID mice from both groups, culled after 21 days post PBMC injection. Quantification of IHC was performed for markers (CD4, CD8 and FOXP3) of human PBMCs that were recruited to human grafts on SCID mice treated with vehicle or the present acidic composition. FIG. 5 provides representative digital images of CD8 stained control grafts (panels a and b) and grafts treated with the acidic composition according to the present methods (panels c and d), demonstrating that the present composition blocks the recruitment of CD8+ cytotoxic T-cells and the anti-inflammatory effect of treatment as described herein. FIG. 6 provides a bar graph showing the % CD8 staining per field in the 10 vehicle-treated (33.50±5.480) and 10 treated (11.20±3.938) groups (labeled “drug”). FIG. 7 provides a bar graph showing the distribution of the number of grafts present in 0-25%, 25-50% and 50-75% staining category in control and treated group.

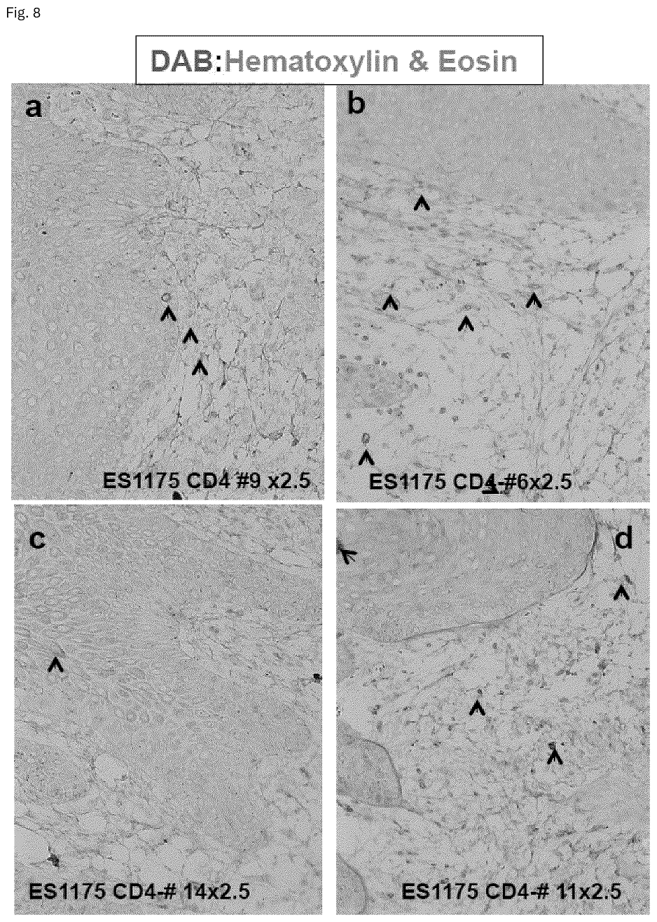

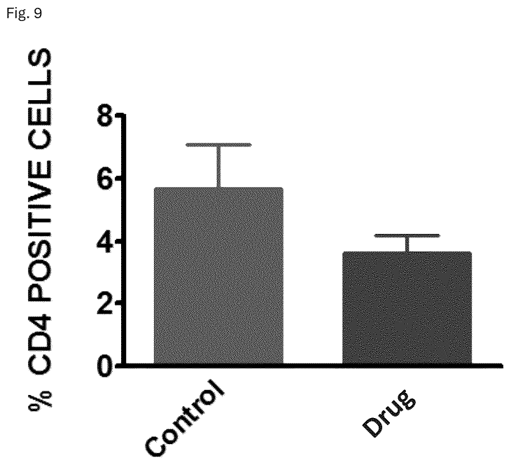

FIG. 8 provides representative digital images of CD4 stained grafts from two controls (panels a and b) and two grafts treated with the present composition (panels c and d) at x2.5 magnification. The arrows in each image denote CD4 positive staining. FIG. 9 provides a bar graph showing the % CD CD4 positive cells in the 6 control (5.672±1.407) and 7 grafts (3.663±0.5359) treated with the present acidic composition (P=0.183).

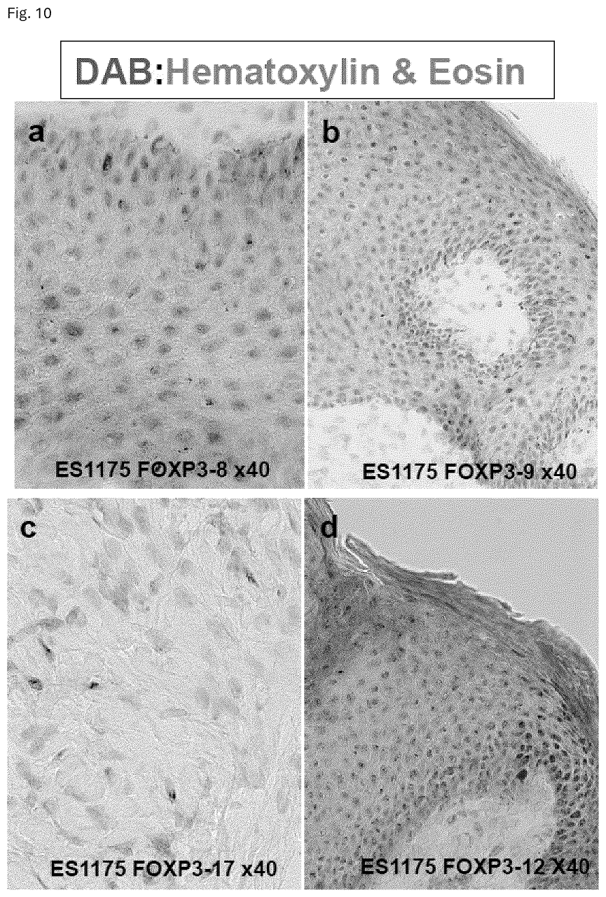

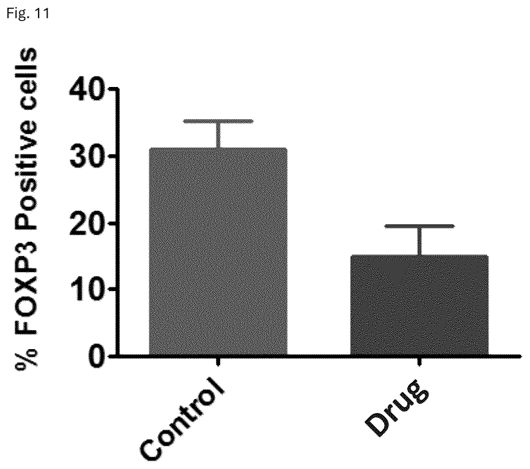

FIG. 10 provides representative digital images of FOXP3 stained grafts from two controls (panels a and b) and two grafts treated with the present composition (panels c and d). FIG. 11 provides a bar graph showing the % FOXP3 positive cells in 5 control (31.08&±&4.063) and 5 treated (14.87&±& 4.729) grafts (P<0.05).

All markers were expressed in both vehicle—as well as treated animals, with some variability in the pattern of staining and percent stained cells between the two groups and within animals of the same group. From the analysis and quantification of the data, we found that percent CD8 staining per field in the vehicle group is 33.50±5.480, (N=10) and lower in the treated group which is 11.20±3.938. (N=10). The decrease observed in the expression of the CD8 marker in the treated group was statistically significantly (p<0.05) compared to the vehicle treated group (See FIG. 6). Similarly, we observed statistically significant decrease (p<0.05) in the expression of percent anti-inflanmatory transcription factor. FOXP3+ cells in the treated group (14.87±4.729, N=5) compared to the Vehicle treated group (31.08±4.063 N=5) (See, FIG. 11).

This example demonstrates a significant reduction of CD8+ cytotoxic T-cells and FOXP3+ inflammatory cells in treated human skin xenografts demonstrating the ability of the present compositions to prevent cutaneous skin inflammation. The reduction of the percent CD3-positive spleenocytes in the treated group demonstrate the effects of the present compositions in the reduction of human inflammatory mediators.

### Example 4 Efficacy of Glycolic Acid:Ethanol Composition in Burn Wound Healing

This example demonstrates the effects of the present compositions on skin burn wound healing and wound repair using 3D-bioprinted human skin (EpiDermFT™):
A. EpiDermFT™ Tissue preparation: Normal primary human dermal fibroblasts (NHDF) were cultured in a collagen gel onto which normal human epidermal keratinocytes were seeded. The constructs were raised to the air/liquid interface and cultured for up to 14 days in serum-free medium to produce stratified, differentiated full-thickness skin equivalents.
B. Tissue and Reagent Storage: EpiDermFI™ inserts (12-24 tissues/kit) were refrigerated in the unopened shipping packages until beginning to culture them. Culture media were stored at 4° C. until use.
C. Tissue Recovery Prior to wounding: Shipped EpiDermFT™ tissues underwent a recovery period prior to wounding, 2.5 ml per well of pre-warmed (37° C.) assay medium was added to each well of four 6-well plates. EpiDermFT™ 3D-skin inserts were transferred from the shipping plates to the 6-well plates containing EpiDermFT™ tissues. The inserts on Day 0: 3D-bioprinted human skin tissues were incubated overnight at 37° C., 5% CO₂, 95% relative humidity individually in pre-warmed (37° C.) 2.5 ml assay medium (without serum) per well in supplied 6-well plates to recover from the shipping conditions and acclimatize in the culture conditions of air-liquid interphase.
Day 1: Burn wounds were induced in each of the 3D skin cultures using a Bovie® low temperature battery powered cautery instrument (Aaron Medical, St. Petersburg, FL). The cautery tip was preheated for 8-10 sec prior to gently contacting the apical surface of the skin tissue for few seconds to induce the burn wound.
Day 1: Two 3D-human skin inserts were treated topically with 10-20 μl vehicle (PBS), two with 0.6% glycolic acid, (pH=2.5), two with human serum added in the medium, three with 0.1 mg/ml of minor cannabinoids, and remaining three were treated with a presently claimed composition (10% ethanol and 0.6% glycolic acid, pH=2.5). Treatment was repeated every 20 minutes, for a total of 3 treatments. The 3× treatment protocol is repeated 4 times a day, for 3.5 consecutive days.
Day 4: To determine the effects of treatment on the healing of human burn wounds and to examine the morphology of the 3D-human skin, the samples were harvested on day 4 post wounding for histopathological analysis.

The inserts containing tissues were split such that ⅔rd was fixed with 4% formalin, embedded in paraffin, and 5 μm thick cross-sections were cut. The sections were mounted on microscopic slides, stained with hematoxylin and eosin (H&E), and observed and photographed using a Lecia bright field microscope.

The remaining ⅓rd skin biopsies were frozen and stored in −80° C. for further analyses at the RNA/protein level.

The epidermal thickness and distance of the migratory tongue of the vehicle and treated samples were measured using the Lecia digital software measured.

The data were graphically represented and analyzed for statistical significance using the Graph Pad Prism software.

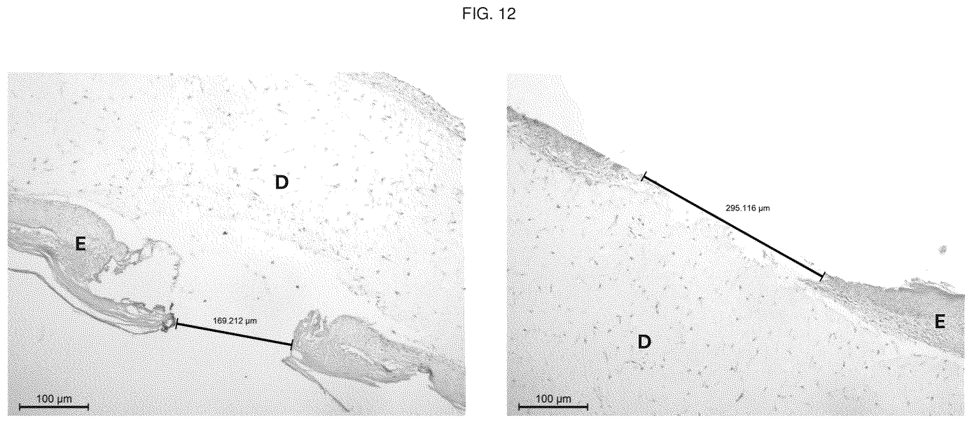

The results of this Example are illustrated in FIGS. 12-16, which demonstrate that topical application of a presently disclosed formulation (10% ethanol and 0.6% glycolic acid, pH-2.5) applied 3× every 20 minutes, 4 times a day for 3.5 days effectively promoted healing of burn wounds in 3D bioprinted human skin compared to the control/vehicle (10% ethanol or PBS). Glycolic acid (pH-2.5) alone was also effective to promote burn wound healing. This example demonstrates that the present compositions are effective to promote healing of burn and chronic wounds in humans.

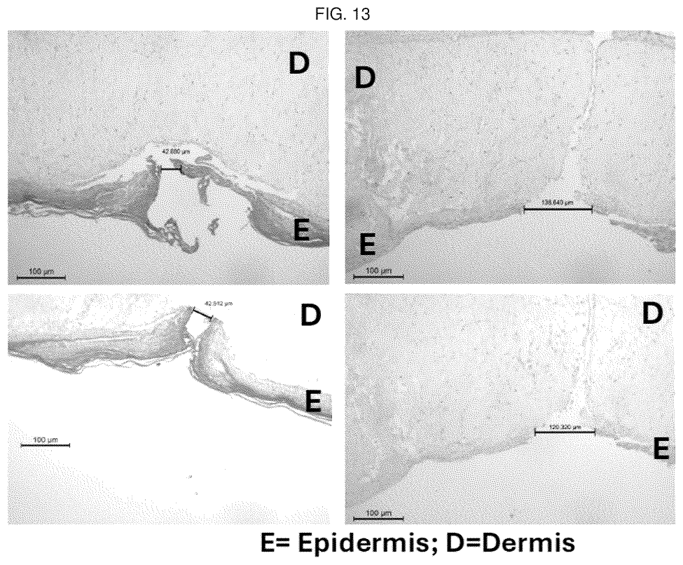

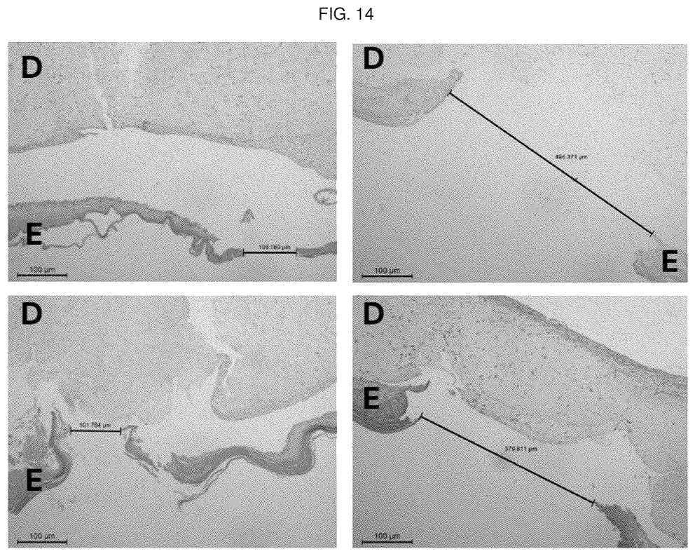

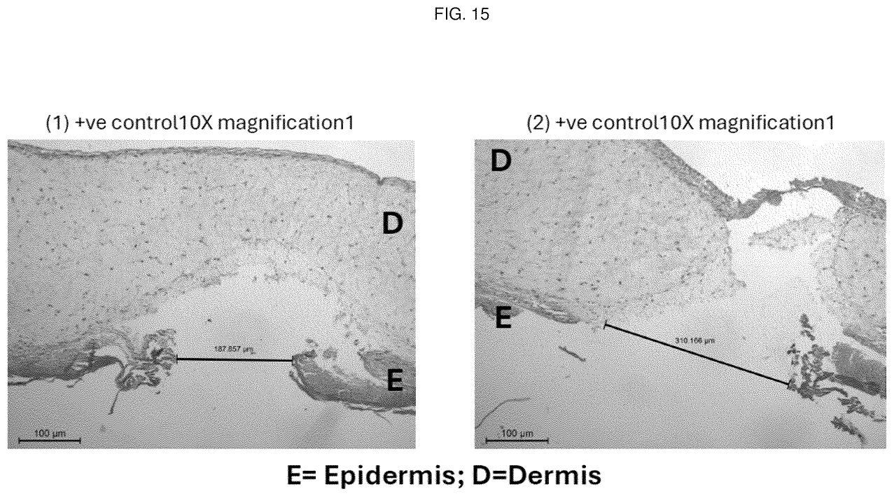

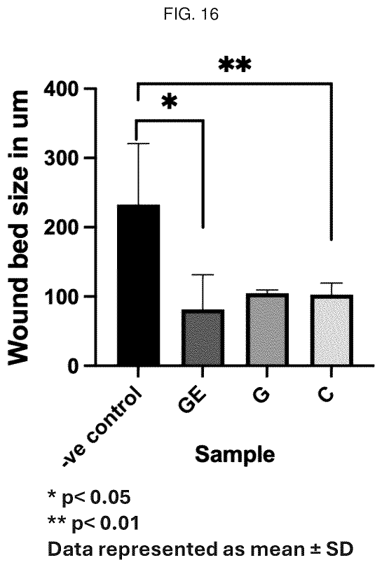

The percentage wound closure for treatment in this example was calculated based on the distance between the migratory tongue in skin burn samples treated with a presently disclosed composition (GE: Glycolic acid 0.6%+10% Ethanol (pH 2.5) as illustrated in FIG. 13; G: Glycolic acid 0.6% (pH 2.5)) as illustrated in FIG. 14 versus the control as illustrated in FIG. 15 and summarized in FIG. 16. Results calculated are that Glycolic acid 0.6%+10% Ethanol (pH 2.5) and Glycolic acid (pH=2.5) increased the rate of burn wound closure by 64.9% (P<0.05) and 54.8%, respectively, over negative control (vehicle only) at day four of treatment.

### REFERENCES

Draper. B. K., Komurasaki, T., Davidson, M. K., and Nanney. L. B. (2003a). Epiregulin is more potent than EGF or TGFalpha in promoting in vitro wound closure due to enhanced ERK/MAPK activation. Journal of cellular biochemistry 89, 1126-1137.
Draper, B. K., Komurasaki, T., Davidson, M. K., and Nanney, L. B. (2003b). Topical epiregulin enhances repair of murine excisional wounds. Wound repair and regeneration: official publication of the Wound Healing Society [and] the European Tissue Repair Society 11, 188-197.
Florin. L., Knebel, L., Zigrino, P., Vonderstrass, B., Mauch, C., Schorpp-Kistner, M., Szabowski, A., and Angel, P. (2006). Delayed wound healing and epidermal hyperproliferation in mice lacking JunB in the skin. The Journal of investigative dermatology 126, 902-911.
Hakkinen, L., Koivisto, L., Gardner, H., Saarialho-Kere, U., Carroll. J. M., Lakso, M., Rauvala, H., Laato, M., IHeino, J., and Larjava, H. (2004). Increased expression of beta6-integrin in skin leads to spontaneous development of chronic wounds. The American journal of pathology 164, 229-242.
Lai, Y., and Gallo, R. L. (2008). Toll-like receptors in skin infections and inflammatory diseases. Infectious disorders drug targets 8, 144-155.
Liang, X., Bhattacharya, S., Bajaj, G., Guha, G., Wang, Z., Jang, H. S., Leid, M., Indra, A. K., and Ganguli-Indra, G. (2012). Delayed cutaneous wound healing and aberrant expression of hair follicle stem cell markers in mice selectively lacking Ctip2 in epidermis. PloS one 7, e29999.
Muehleisen, B., Bikle, D. D., Aguilera. C., Burton. D. W., Sen. G. L., Deftos. L. J., and Gallo, R. L. (2012). PTH/PTHrP and vitamin D control antimicrobial peptide expression and susceptibility to bacterial skin infection. Science translational medicine 4, 135ra166.
Nizet, V., Ohtake, T.. [auth, X., Trowbridge, J., Rudisill, J., Dorschner, R. A., Pestonjamasp, V., Piraino, J., Huttner, K., and Gallo, R. L. (2001). Innate antimicrobial peptide protects the skin from invasive bacterial infection. Nature 414, 454-457.
Zhang, L. J., Guerrero-Juarez, C. F., Hata, T., Bapat. S. P., Ramos, R., Plikus, M. V., and Gallo, R. L. (2015). Innate immunity. Dermal adipocytes protect against invasive Staphylococcus aureus skin infection. Science 347, 67-71.

### Example 5 Assessment of Antimicrobial Efficacy of Glycolic Acid:Ethanol Composition

This example describes the use of the Kirby-Bauer disk diffusion susceptibility test to determine the sensitivity or resistance of pathogenic aerobic and facultative anaerobic bacteria to the present antimicrobial compositions. This test can be used to assist a physician in selecting treatment options for their patients. The pathogenic organism is grown on Mueller-Hinton agar in the presence of various antimicrobial impregnated filter paper disks. The presence or absence of growth around the disks is an indirect measure of the ability of that composition to inhibit growth of that organism.

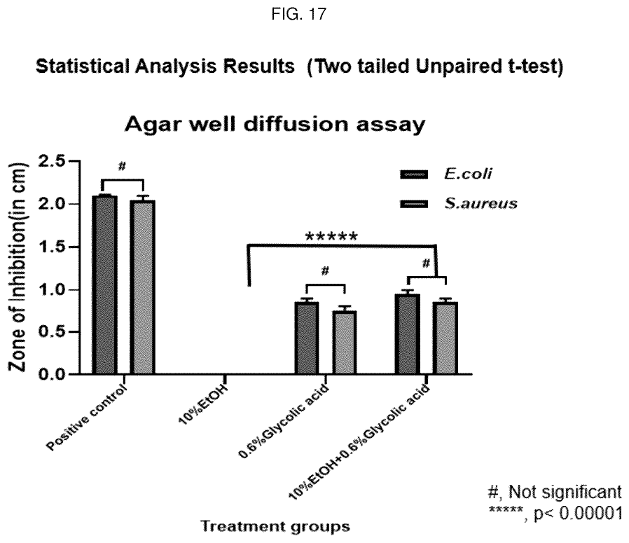

The E. coli and S. aureus were grown on Mueller-Hinton agar in the presence of various filter paper disks treated with a positive control (Apramycin(1 mg/ml); 10% ethanol, 0.6% glycolic acid; or 10% ethanol/0.6% glycolic acid solution. The growth around the disks was measured and recorded in the Table below. A summary of the results is presented in the bar graph of FIG. 17.

| | | E. coli | | | S. aureus | | |
|---|---|---|---|---|---|---|---|
| S. NO | Groups | Replica-1 | Replica-2 | Replica-3 | Replica-1 | Replica-2 | Replica-3 |
| 1 | Positive | 2.1 cm | 2.1 cm | 2.1 cm | 2 cm | 2 cm | 2 cm |
| | control(Apramycin(1 mg/ml) | | | | | | |
| 2 | 10% EtOH | No ZOI | No ZOI | No ZOI | No ZOI | No ZOI | No ZOI |
| 3 | 0.6% Glycolic acid | 0.8 cm | 0.8 cm | 0.8 cm | 0.7 cm | 0.7 cm | 0.7 cm |
| 4 | 10% EtOH + 0.6% | 0.9 cm | 0.9 cm | 0.9 cm | 0.8 cm | 0.8 cm | 0.8 cm |
| | Glycolic acid | | | | | | |

With continued reference to the Table above and FIG. 17, 10% ethanol exhibited no Zone of Inhibition (ZOI), whereas 0.6% glycolic acid and 10% ethanol/0.6% glycolic acid solutions both demonstrated inhibition of both E. coli and S. aureus.

### REFERENCES

1. Bauer, A. W., D. M. Perry, and W. M. M. Kirby. 1959. Single disc antibiotic sensitivity testing of Staphylococci. A. M. A. Arch. Intern. Med. 104:208-216.
2. Bauer, A. W., W. M. M. Kirby, J. C. Sherris, and M. Turck. 1966. Antibiotic susceptibility testing by a standardized single disk method. Am. J. Clin. Pathol. 36:493-496.
3. Clinical Laboratory Standards Institute. 2006. Performance standards for antimicrobial disk susceptibility tests; Approved standard—9th ed. CLSI document M2-A9, 26:1. Clinical Laboratory Standards Institute, Wayne, PA.
4. Difco. 1984. Difco manual, 10th ed. Difco Laboratories, Detroit, MI.
5. Kirby, W. M. M., G. M. Yoshihara, K. S. Sundsted, and J. H. Warren. 1957. Clinical usefulness of a single disc method for antibiotic sensitivity testing. Antibiotics Annu. 1956-1957:892.
6. Winn, Jr., W., et al. 2006. Konemann's color atlas and diagnostic text of microbiology, 6th ed., p. 945-1021. Lippencott Williams & Wilkins Publishers, Philadelphia, PA.
7. Jorgensen. J. H., and J. D. Turnidge. 2007. Susceptibility test methods: dilution and disk diffusion methods, p. 1152-1172. In P. R. Murray, E. J. Baron. J. H. Jorgensen, M. L. Landry, and M. A. Pfaller (ed.). Manual of clinical microbiology, 9th ed. ASM Press, Washington, D.C.
8. Hudzicki, Jan. 2009 Kirby-Bauer Disk Diffusion Susceptibility Test Protocol. ASM, 2016

1. A method of reducing or decreasing a lesion on a surface of a subject comprising
contacting the lesion and/or an area of the surface adjacent to the lesion with an effective amount of a composition comprising a C1-C10 alcohol and a sufficient amount of an acid to adjust the pH to below 5.5, wherein the lesion is not caused by a virus.

2. The method of claim 1, wherein the lesion comprises a gash, cut, puncture, abrasion, bump, blister, ulcer, allergic reaction, or burn on the surface.

3. The method of claim 1, wherein the lesion is the result of an insect, an arachnid, or an animal bite or sting.

4. The method of claim 3, wherein the animal is a mammal, a reptile, an amphibian, a cnidarian, or a mollusk.

5. The method of claim 1, wherein the lesion is caused by a bacterium or fungus.

6. The method of claim 1, wherein the lesion is caused by contact with a plant, tree.

7. The method of claim 1, wherein the lesion is caused by contact with a chemical irritant.

8. The method of claim 1, wherein the lesion is a benign lesion, a pre-cancerous lesion, or a cancerous lesion.

9. The method of claim 1, wherein the surface is selected from the group consisting of: a skin surface, a surface of a mucous membrane surface, and a surface of a nail.

10. The method of claim 1, wherein the C1-C10 alcohol is a C1 to C3 monohydroxy alcohol or a C1 to C4 diol, and the acid is an organic acid.

11. The method of claim 10, wherein the alcohol is selected from the group consisting of methanol, ethanol, 1-propanol, and 2-propanol, and the organic acid comprises glycolic acid.

12. A method of reducing or decreasing inflammation of a surface of a subject comprising contacting the inflamed surface with an effective amount of a composition comprising a C1-C10 alcohol and a sufficient amount of an acid to adjust the pH to below 5.5, wherein the lesion is not caused by a virus.

13. The method of claim 12, wherein the inflammation is the result of a lesion, an insect bite or sting, dermatitis, drug reaction, psoriasis or interstitial cystitis.

14. The method of claim 12, wherein the inflamed surface is selected from the group consisting of: a skin surface, a surface of a mucous membrane, and a surface of a nail.

15. The method of claim 12, wherein the wherein the C1-C10 alcohol is a C1 to C3 monohydroxy alcohol or a C1 to C4 diol, and the acid is an organic acid.

16. The method of claim 15, wherein the C1 to C3 monohydroxy alcohol is ethanol and the organic acid is glycolic acid.

17. A method for treating an inflammatory or autoimmune lesion, comprising contacting the lesion with an effective amount of an acidic composition comprising a C1-C10 alcohol.

18. The method according to claim 1, wherein the alcohol concentration in the composition ranges from about 1% by volume up to about 15% by volume; and
the glycolic acid concentration in the composition ranges from about about 0.5% w/v to about 1% w/v glycolic acid.

19. The method of claim 17, wherein the acidic composition comprises an amount of an acid sufficient to provide a pH of less than 5.5.

20. The method of claim 17, wherein the inflammatory or autoimmune lesion is associate with a disorder selected from psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne and alopecia areata.
